SEQUENCE LISTING (24 sequences)

SEQ ID NO: 1 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYFLVADDMMDKSITRRGQPCWYKVPEVGEIAINDAFMLEAAIYKLLKSHFRNEKYYIDITELFHEVTFQTELGQLMDLITAPEDKVDLSKFSLKKHSFIVTFETAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 2 - protein, 352 aa, Saccharomyces cerevisiae
MASEKEIRRERFLNVFPKLVEELNASLLAYGMPKEACDWYAHSLNYNTPGGKLNRGLSVVDTYAILSNKTVEQLGQEEYEKVAILGWCIELLQAYWLVADDMMDKSITRRGQPCWYKVPEVGEIAIWDAFMLEAAIYKLLKSHFRNEKYYIDITELFHEVTFQTELGQLMDLITAPEDKVDLSKFSLKKHSFIVTFKTAYYSFYLPVALAMYVAGITDEKDLKQARDVLIPLGEYFQIQDDYLDCFGTPEQIGKIGTDIQDNKCSWVINKALELASAEQRKTLDENYGKKDSVAEAKCKKIFNDLKIEQLYHEYEESIAKDLKAKISQVDESRGFKADVLTAFLNKVYKRSK

SEQ ID NO: 3 - protein, 344 aa, Yarrowia lipolytica
MSKAKFESVFPRISEELVQLLRDEGLPQDAVQWFSDSLQYNCVGGKLNRGLSVVDTYQLLTGKKELDDEEYYRLALLGWLIELLQAFFLVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYETAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 4 - protein, 344 aa, Yarrowia lipolytica
ASKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 5 - protein, 999 aa, Yarrowia lipolytica
MLQAAIGKIVGFAVNRPIHTVVLTSIVASTAYLAILDIAIPGFEGTQPISYYHPAAKSYDNPADWTHIAEADIPSDAYRLAFAQIRVSDVQGGEAPTIPGAVAVSDLDHRIVMDYKQWAPWTASNEQIASENHIWKHSFKDHVAFSWIKWFRWAYLRLSTLIQGADNFDIAVVALGYLAMHYTFFSLFRSMRKVGSHFWLASMALVSSTFAFLLAVVASSSLGYRPSMITMSEGLPFLVVAIGFDRKVNLASEVLTSKSSQLAPMVQVITKIASKALFEYSLEVAALFAGAYTGVPRLSQFCFLSAWILIFDYMFLLTFYSAVLAIKFEINHIKRNRMIQDALKEDGVSAAVAEKVADSSPDAKLDRKSDVSLFGASGAIAVFKIFMVLGFLGLNLINLTAIPHLGKAAAAAQSVTPITLSPELLHAIPASVPVVVTFVPSVVYEHSQLILQLEDALTTFLAACSKTIGDPVISKYIFLCLMVSTALNVYLFGATREVVRTQSVKVVEKHVPIVIEKPSEKEEDTSSEDSIELTVGKQPKPVTETRSLDDLEAIMKAGKTKLLEDHEVVKLSLEGKLPLYALEKQLGDNTRAVGIRRSIISQQSNTKTLETSKLPYLHYDYDRVFGACCENVIGYMPLPVGVAGPMNIDGKNYHIPMATTEGCLVASTMRGCKAINAGGGVTTVLTQDGMTRGPCVSFPSLKRAGAAKIWLDSEEGLKSMRKAFNSTSRFARLQSLHSTLAGNLLFIRFRTTTGDAMGMNMISKGVEHSLAVMVKEYGFPDMDIVSVSGNYCTDKKPAAINWIEGRGKSVVAEATIPAHIVKSVLKSEVDALVELNISKNLIGSAMAGSVGGFNAHAANLVTAIYLATGQDPAQNVESSNCITLMSNVDGNLLISVSMPSIEVGTIGGGTILEPQGAMLEMLGVRGPHIETPGANAQQLARIIASGVLAAELSLCSALAAGHLVQSHMTHNRSQAPTPAKQSQADLQRLQNGSNICIRS

SEQ ID NO: 6 - protein, 499 aa, Yarrowia lipolytica
TQSVKVVEKHVPIVIEKPSEKEEDTSSEDSIELTVGKQPKPVTETRSLDDLEAIMKAGKTKLLEDHEVVKLSLEGKLPLYALEKQLGDNTRAVGIRRSIISQQSNTKTLETSKLPYLHYDYDRVFGACCENVIGYMPLPVGVAGPMNIDGKNYHIPMATTEGCLVASTMRGCKAINAGGGVTTVLTQDGMTRGPCVSFPSLKRAGAAKIWLDSEEGLKSMRKAFNSTSRFARLQSLHSTLAGNLLFIRFRTTTGDAMGMNMISKGVEHSLAVMVKEYGFPDMDIVSVSGNYCTDKKPAAINWIEGRGKSVVAEATIPAHIVKSVLKSEVDALVELNISKNLIGSAMAGSVGGFNAHAANLVTAIYLATGQDPAQNVESSNCITLMSNVDGNLLISVSMPSIEVGTIGGGTILEPQGAMLEMLGVRGPHIETPGANAQQLARIIASGVLAAELSLCSALAAGHLVQSHMTHNRSQAPTPAKQSQADLQRLQNGSNICIRS

SEQ ID NO: 7 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 8 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 9 - protein, 395 aa, Cannabis sativa
MGLSSVCTFSFQTNYHTLLNPHNNNPKTSLLCYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 10 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 11 - protein, 400 aa, Cannabis sativa
MVFSSVCSFPSSLGTNFKLVPRSNFKASSSHYHEINNFINNKPIKFSYFSSRLYCSAKPIVHRENKFTKSFSLSHLQRKSSIKAHGEIEADGSNGTSEFNVMKSGNAIWRFVRPYAAKGVLFNSAAMFAKELVGNLNLFSWPLMFKILSFTLVILCIFVSTSGINQIYDLDIDRLNKPNLPVASGEISVELAWLLTIVCTISGLTLTIITNSGPFFPFLYSASIFFGFLYSAPPFRWKKNPFTACFCNVMLYVGTSVGVYYACKASLGLPANWSPAFCLLFWFISLLSIPISIAKDLSDIEGDRKFGIITFSTKFGAKPIAYICHGLMLLNYVSVMAAAIIWPQFFNSSVILLSHAFMAIWVLYQAWILEKSNYATETCQKYYIFLWIIFSLEHAFYLFM

SEQ ID NO: 12 - protein, 307 aa, Streptomyces sp. CL190
MSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVAMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAYYHITDVQRGLLKAFDSLED

SEQ ID NO: 13 - protein, 517 aa, Cannabis sativa
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 14 - protein, 516 aa, Cannabis sativa
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - protein, 517 aa, Cannabis sativa
NPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAAWKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 16 - protein, 1671 aa, Aspergillus parasiticus
MVIQGKRLAASSIQLLASSLDAKKLCYEYDERQAPGVTQITEEAPTEQPPLSTPPSLPQTPNISPISASKIVIDDVALSRVQIVQALVARKLKTAIAQLPTSKSIKELSGGRSSLQNELVGDIHNEFSSIPDAPEQILLRDFGDANPTVQLGKTSSAAVAKLISSKMPSDFNANAIRAHLANKWGLGPLRQTAVLLYAIASEPPSRLASSSAAEEYWDNVSSMYAESCGITLRPRQDTMNEDAMASSAIDPAVVAEFSKGHRRLGVQQFQALAEYLQIDLSGSQASQSDALVAELQQKVDLWTAEMTPEFLAGISPMLDVKKSRRYGSWWNMARQDVLAFYRRPSYSEFVDDALAFKVFLNRLCNRADEALLNMVRSLSCDAYFKQGSLPGYHAASRLLEQAITSTVADCPKARLILPAVGPHTTITKDGTIEYAEAPRQGVSGPTAYIQSLRQGASFIGLKSADVDTQSNLTDALLDAMCLALHNGISFVGKTFLVTGAGQGSIGAGVVRLLLEGGARVLVTTSREPATTSRYFQQMYDNHGAKFSELRVVPCNLASAQDCEGLIRHVYDPRGLNWDLDAILPFAAASDYSTEMHDIRGQSELGHRLMLVNVFRVLGHIVHCKRDAGVDCHPTQVLLPLSPNHGIFGGDGMYPESKLALESLFHRIRSESWSDQLSICGVRIGWTRSTGLMTAHDIIAETVEEHGIRTFSVAEMALNIAMLLTPDFVAHCEDGPLDADFTGSLGTLGSIPGFLAQLHQKVQLAAEVIRAVQAEDEHERFLSPGTKPTLQAPVAPMHPRSSLRVGYPRLPDYEQEIRPLSPRLERLQDPANAVVVVGYSELGPWGSARLRWEIESQGQWTSAGYVELAWLMNLIRHVNDESYVGWVDTQTGKPVRDGEIQALYGDHIDNHTGIRPIQSTSYNPERMEVLQEVAVEEDLPEFEVSQLTADAMRLRHGANVSIRPSGNPDACHVKLKRGAVILVPKTVPFVWGSCAGELPKGWTPAKYGIPENLIHQVDPVTLYTICCVAEAFYSAGITHPLEVFRHIHLSELGNFIGSSMGGPTKTRQLYRDVYFDHEIPSDVLQDTYLNTPAAWVNMLLLGCTGPIKTPVGACATGVESIDSGYESIMAGKTKMCLVGGYDDLQEEASYGFAQLKATVNVEEEIACGRQPSEMSRPMAESRAGFVEAHGCGVQLLCRGDIALQMGLPIYAVIASSAMAADKIGSSVPAPGQGILSFSRERARSSMISVTSRPSSRSSTSSEVSDKSSLTSITSISNPAPRAQRARSTTDMAPLRAALATWGLTIDDLDVASLHGTSTRGNDLNEPEVIETQMRHLGRTPGRPLWAICQKSVTGHPKAPAAAWMLNGCLQVLDSGLVPGNRNLDTLDEALRSASHLCFPTRTVQLREVKAFLLTSFGFGQKGGQVVGVAPKYFFATLPRPEVEGYYRKVRVRTEAGDRAYAAAVMSQAVVKIQTQNPYDEPDAPRIFLDPLARISQDPSTGQYRFRSDATPALDDDALPPPGEPTELVKGISSAWIEEKVRPHMSPGGTVGVDLVPLASFDAYKNAIFVERNYTVRERDWAEKSADVRAAYASRWCAKEAVFKCLQTHSQGAGAAMKEIEIEHGGNGAPKVKLRGAAQTAARQRGLEGVQLSISYGDDAVIAVALGLMSGAS

SEQ ID NO: 17 - protein, 1888 aa, Aspergillus parasiticus
MGSVSREHESIPIQAAQRGAARICAAFGGQGSNNLDVLKGLLELYKRYGPDLDELLDVASNTLSQLASSPAAIDVHEPWGFDLRQWLTTPEVAPSKEILALPPRSFPLNTLLSLALYCATCRELELDPGQFRSLLHSSTGHSQGILAAVAITQAESWPTFYDACRTVLQISFWIGLEAYLFTPSSAASDAMIQDCIEHGEGLLSSMLSVSGLSRSQVERVIEHVNKGLGECNRWVHLALVNSHEKFVLAGPPQSLWAVCLHVRRIRADNDLDQSRILFRNRKPIVDILFLPISAPFHTPYLDGVQDRVIEALSSASLALHSIKIPLYHTGTGSNLQELQPHQLIPTLIRAITVDQLDWPLVCRGLNATHVLDFGPGQTCSLIQELTQGTGVSVIQLTTQSGPKPVGGHLAAVNWEAEFGLRLHANVHGAAKLHNRMTTLLGKPPVMVAGMTPTTVRWDFVAAVAQAGYHVELAGGGYHAERQFEAEIRRLATAIPADHGITCNLLYAKPTTFSWQISVIKDLVRQGVPVEGITIGAGIPSPEVVQECVQSIGLKHISFKPGSFEAIHQVIQIARTHPNFLIGLQWTAGRGGGHHSWEDFHGPILATYAQIRSCPNILLVVGSGFGGGPDTFPYLTGQWAQAFGYPCMPFDGVLLGSRMMVAREAHTSAQAKRLIIDAQGVGDADWHKSFDEPTGGVVTVNSEFGQPIHVLATRGVMLWKELDNRVFSIKDTSKRLEYLRNHRQEIVSRLNADFARPWFAVDGHGQNVELEDMTYLEVLRRLCDLTYVSHQKRWVDPSYRILLLDFVHLLRERFQCAIDNPGEYPLDIIVRVEESLKDKAYRTLYPEDVSLLMHLFSRRDIKPVPFIPRLDERFETWFKKDSLWQSEDVEAVIGQDVQRIFIIQGPMAVQYSISDDESVKDILHNICNHYVEALQADSRETSIGDVHSITQKPLSAFPGLKVTTNRVQGLYKFEKVGAVPEMDVLFEHIVGLSKSWARTCLMSKSVFRDGSRLHNPIRAALQLQRGDTIEVLLTADSEIRKIRLISPTGDGGSTSKVVLEIVSNDGQRVFATLAPNIPLSPEPSVVFCFKVDQKPNEWTLEEDASGRAERIKALYMSLWNLGFPNKASVLGLNSQFTGEELMITTDKIRDFERVLRQTSPLQLQSWNPQGCVPIDYCVVIAWSALTKPLMVSSLKCDLLDLLHSAISFHYAPSVKPLRVGDIVKTSSRILAVSVRPRGTMLTVSADIQRQGQHVVTVKSDFFLGGPVLACETPFELTEEPEMVVHVDSEVRRAILHSRKWLMREDRALDLLGRQLLFRLKSEKLFRPDGQLALLQVTGSVFSYSPDGSTTAFGRVYFESESCTGNVVMDFLHRYGAPRAQLLELQHPGWTGTSTVAVRGPRRSQSYARVSLDHNPIHVCPAFARYAGLSGPIVHGMETSAMMRRIAEWAIGDADRSRFRSWHITLQAPVHPNDPLRVELQHKAMEDGEMVLKVQAFNERTEERVAEADAHVEQETTAYVFCGQGSQRQGMGMDLYVNCPEAKALWARADKHLWEKYGFSILHIVQNNPPALTVHFGSQRGRRIRANYLRMMGQPPIDGRHPPILKGLTRNSTSYTFSYSQGLLMSTQFAQPALALMEMAQFEWLKAQGVVQKGARFAGHSLGEYAALGACASFLSFEDLISLIFYRGLKMQNALPRDANGHTDYGMLAADPSRIGKGFEEASLKCLVHIIQQETGWFVEVVNYNINSQQYVCAGHFRALWMLGKICDDLSCHPQPETVEGQELRAMVWKHVPTVEQVPREDRMERGRATIPLPGIDIPYHSTMLRGEIEPYREYLSERIKVGDVKPCELVGRWIPNVVGQPFSVDKSYVQLVHGITGSPRLHSLLQQMA

SEQ ID NO: 18 - protein, 1559 aa, Aspergillus nidulans
MTQKTIQQVPRQGLELLASTQDLAQLCYIYGEPAEGEDSTADESIINTPQCSTIPEVAVEPEVQPIPDTPLTAIFIIRALVARKLRRSETEIDPSRSIKELCGGKSTLQNELIGELGNEFQTSLPDRAEDVSLADLDAALGEVSLGPTSVSLLQRVFTAKMPARMTVSNVRERLAEIWGLGFHRQTAVLVAALAAEPHSRLTSLEAAYQYWDGLNEAYGQSLGLFLRKAISQQAARSDDQGAQAIAPADSLGSKDLARKQYEALREYLGIRTPTTKQDGLDLADLQQKLDCWTAEFSDDFLSQISRRFDARKTRWYRDWWNSARQELLTICQNSNVQWTDKMREHFVQRAEEGLVEIARAHSLAKPLVPDLIQAISLPPVVRLGRLATMMPRTVVTLKGEIQCEEHEREPSCFVEFFSSWIQANNIRCTIQSNGEDLTSVFINSLVHASQQGVSFPNHTYLITGAGPGSIGQHIVRRLLTGGARVIVTTSREPLPAAAFFKELYSKCGNRGSQLHLVPFNQASVVDCERLIGYIYDDLGLDLDAILPFAATSQVGAEIDGLDASNEAAFRLMLVNVLRLVGFVVSQKRRRGISCRPTQVVLPLSPNHGILGGDGLYAESKRGLETLIQRFHSESWKEELSICGVSIGWTRSTGLMAANDLVAETAEKQGRVLTFSVDEMGDLISLLLTPQLATRCEDAPVMADFSGNLSCWRDASAQLAAARASLRERADTARALAQEDEREYRCRRAGSTQEPVDQRVSLHLGFPSLPEYDPLLHPDLVPADAVVVVGFAELGPWGSARIRWEMESRGCLSPAGYVETAWLMNLIRHVDNVNYVGWVDGEDGKPVADADIPKRYGERILSNAGIRSLPSDNREVFQEIVLEQDLPSFETTRENAEALQQRHGDMVQVSTLKNGLCLVQLQHGATIRVPKSIMSPPGVAGQLPTGWSPERYGIPAEIVQQVDPVALVLLCCVAEAFYSAGISDPMEIFEHIHLSELGNFVGSSMGGVVNTRALYHDVCLDKDVQSDALQETYLNTAPAWVNMLYLGAAGPIKTPVGACATALESVDSAVESIKAGQTKICLVGGYDDLQPEESAGFARMKATVSVRDEQARGREPGEMSRPTAASRSGFVESQGCGVQLLCRGDVALAMGLPIYGIIAGTGMASDGIGRSVPAPGQGILTFAQEDAQNPAPSRTALARWGLGIDDITVASLHATSTPANDTNEPLVIQREMTHLGRTSGRPLWAICQKFVTGHPKAPAAAWMLNGCLQVLDTGLVPGNRNADDVDPALRSFSHLCFPIRSIQTDGIKAFLLNSCGFGQKEAQLVGVHPRYFLGLLSEPEFEEYRTRRQLRIAGAERAYISAMMTNSIVCVQSHPPFGPAEMHSILLDPSARICLDSSTNSYRVTKASTPVYTGFQRPHDKREDPRPSTIGVDTVTLSSFNAHENAIFLQRNYTERERQSLQLQSHRSFRSAVASGWCAKEAVFKCLQTVSKGAGAAMSEIEIVRVQGAPSVLHGDALAAAQKAGLDNIQLSLSYGDDCVVAVALGVRKWCLWPLASIIR

SEQ ID NO: 19 - protein, 1914 aa, Aspergillus nidulans
MTPSPFLDAVDAGLSRLYACFGGQGPSNWAGLDELVHLSHAYADCAPIQDLLDSSARRLESQQRSHTDRHFLLGAGSNYRPGSTTLLHPHHLPEDLALSPYSFPINTLLSLLHYAITAYSLQLDPGQLRQKLQGAIGHSQGVFVAAAIAISHTDHGWPSFYRAADLALQLSFWVGLESHHASPRSILCANEVIDCLENGEGAPSHLLSVTGLDINHLERLVRKLNDQGGDSLYISLINGHNKFVLAGAPHALRGVCIALRSVKASPELDQSRVPFPLRRSVVDVQFLPVSAPYHSSLLSSVELRVTDAIGGLRLRGNDLAIPVYCQANGSLRNLQDYGTHDILLTLIQSVTVERVNWPALCWAMNDATHVLSFGPGAVGSLVQDVLEGTGMNVVNLSGQSMASNLSLLNLSAFALPLGKDWGRKYRPRLRKAAEGSAHASIETKMTRLLGTPHVMVAGMTPTTCSPELVAAIIQADYHVEFACGGYYNRATLETALRQLSRSIPPHRSITCNVIYASPKALSWQTQVLRRLIMEEGLPIDGITVGAGIPSPEVVKEWIDMLAISHIWFKPGSVDAIDRVLTIARQYPTLPVGIQWTGGRAGGHHSCEDFHLPILDCYARIRNCENVILVAGSGFGGAEDTWPYMNGSWSCKLGYAPMPFDGILLGSRMMVAREAKTSFAVKQLIVEAPGVKDDGNDNGAWAKCEHDAVGGVISVTSEMGQPIHVLATRAMRLWKEFDDRFFSIRDPKRLKAALKQHRVEIINRLNNDFARPWFAQTDSSKPTEIEELSYRQVLRRLCQLTYVQHQARWIDSSYLSLVHDFLRLAQGRLGSGSEAELRFLSCNTPIELEASFDAAYGVQGDQILYPEDVSLLINLFRRQGQKPVPFIPRLDADFQTWFKKDSLWQSEDVDAVVDQDAQRVCIIQGPVAVRHSRVCDEPVKDILDGITEAHLKMMLKEAASDNGYTWANQRDEKGNRLPGIETSQEGSLCRYYLVGPTLPSTEAIVEHLVGECAWGYAALSQKKVVFGQNRAPNPIRDAFKPDIGDVIEAKYMDGCLREITLYHSLRRQGDPRAIRAALGLIHLDGNKVSVTLLTRSKGKRPALEFKMELLGGTMGPLILKMHRTDYLDSVRRLYTDLWIGRDLPSPTSVGLNSEFTGDRVTITAEDVNTFLAIVGQAGPARCRAWGTRGPVVPIDYAVVIAWTALTKPILLEALDADPLRLLHQSASTRFVPGIRPLHVGDTVTTSSRITERTITTIGQRVEISAELLREGKPVVRLQTTFIIQRRPEESVSQQQFRCVEEPDMVIRVDSHTKLRVLMSRKWFLLDGPCSDLIGKILIFQLHSQTVFDAAGAPASLQVSGSVSLAPSDTSVVCVSSVGTRIGRVYMEEEGFGANPVMDFLNRHGAPRVQRQPLPRAGWTGDDAASISFTAPAQSEGYAMVSGDTNPIHVCPLFSRFAGLGQPVVHGLHLSATVRRILEWIIGDNERTRFCSWAPSFDGLVRANDRLRMEIQHFAMADGCMVVHVRVLKESTGEQVMHAEAVLEQAQTTYVFTGQGTQERGMGMALYDTNAAARAVWDRAERHFRSQYGISLLHIVRENPTSLTVNFGSRRGRQIRDIYLSMSDSDPSMLPGLTRDSRSYTFNYPSGLLMSTQFAQPALAVMEIAEYAHLQAQGVVQTQAIFAGHSLGEYSSLGACTTIMPFESLLSLILYRGLKMQNTLPRNANGRTDYGMVAADPSRIRSDFTEDRLIELVRLVSQATGVLLEVVNYNVHSRQYVCAGHVRSLWVLSHACDDLSRSTSPNSPQTMSECIAHHIPSSCSVTNETELSRGRATIPLAGVDIPFHSQMLRGHIDGYRQYLRHHLRVSDIKPEELVGRWIPNVTGKPFALDAPYIRLVQGVTQSRPLLELLRRVEENR

SEQ ID NO: 20 - protein, 1858 aa, Yarrowia lipolytica
MRPEIEQELAHTLLVELLAYQFASPVRWIETQDVILAEKRTERIVEIGPADTLGGMARRTLASKYEAYDAATSVQRQILCYNKDAKEIYYDVDPVEEETESAPEAAAAPPTSAAPAAAVVAAPAPAASAPSAGPAAPVEDAPVTALDIVRTLVAQKLKKALSDVPLNKAIKDLVGGKSTLQNEILGDLGKEFGSTPEKPEDTPLDELGASMQATFNGQLGKQSSSLIARLVSSKMPGGFNITAVRKYLETRWGLGPGRQDGVLLLALTMEPASRIGSEPDAKVFLDDVANKYAANSGISLNVPTASGDGGASAGGMLMDPAAIDALTKDQRALFKQQLEIIARYLKMDLRDGQKAFVASQETQKTLQAQLDLWQAEHGDFYASGIEPSFDPLKARVYDSSWNWARQDALSMYYDIIFGRLKVVDREIVSQCIRIMNRSNPLLLEFMQYHIDNCPTERGETYQLAKELGEQLIENCKEVLGVSPVYKDVAVPTGPQTTIDARGNIEYQEVPRASARKLEHYVKQMAEGGPISEYSNRAKVQNDLRSVYKLIRRQHRLSKSSQLQFNALYKDVVRALSMNENQIMPQENGSTKKPGRNGSVRNGSPRAGKVETIPFLHLKKKNEHGWDYSKKLTGIYLDVLESAARSGLTFQGKNVLMTGAGAGSIGAEVLQGLISGGAKVIVTTSRYSREVTEYYQAMYARYGARGSQLVVVPFNQGSKQDVEALVDYIYDTKKGLGWDLDFIVPFAAIPENGREIDSIDSKSELAHRIMLTNLLRLLGSVKAQKQANGFETRPAQVILPLSPNHGTFGNDGLYSESKLALETLFNRWYSENWSNYLTICGAVIGWTRGTGLMSGNNMVAEGVEKLGVRTFSQQEMAFNLLGLMAPAIVNLCQLDPVWADLNGGLQFIPDLKDLMTRLRTEIMETSDVRRAVIKETAIENKVVNGEDSEVLYKKVIAEPRANIKFQFPNLPTWDEDIKPLNENLKGMVNLDKVVVVTGFSEVGPWGNSRTRWEMEASGKFSLEGCVEMAWIMGLIRHHNGPIKGKTYSGWVDSKTGEPVDDKDVKAKYEKYILEHSGIRLIEPELFKGYDPKKKQLLQEIVIEEDLEPFEASKETAEEFKREHGEKVEIFEVLESGEYTVRLKKGATLLIPKALQFDRLVAGQVPTGWDARRYGIPEDIIEQVDPVTLFVLVCTAEAMLSAGVTDPYEFYKYVHLSEVGNCIGSGIGGTHALRGMYKDRYLDKPLQKDILQESFINTMSAWVNMLLLSSTGPIKTPVGACATAVESVDIGYETIVEGKARVCFVGGFDDFQEEGSYEFANMKATSNAEDEFAHGRTPQEMSRPTTTTRAGFMESQGCGMQLIMSAQLALDMGVPIYGIIALTTTATDKIGRSVPAPGQGVLTTARENPGKFPSPLLDIKYRRRQLELRKRQIREWQESELLYLQEEAEAIKAQNPADFVVEEYLQERAQHINREAIRQEKDAQFSLGNNFWKQDSRIAPLRGALATWGLTVDEIGVASFHGTSTVANDKNESDVICQQMKHLGRKKGNALLGIFQKYLTGHPKGAAGAWMFNGCLQVLDSGLVPGNRNADNVDKVMEKFDYIVYPSRSIQTDGIKAFSVTSFGFGQKGAQVIGIHPKYLYATLDRAQFEAYRAKVETRQKKAYRYFHNGLVNNSIFVAKNKAPYEDELQSKVFLNPDYRVAADKKTSELKYPPKPPVATDAGSESTKAVIESLAKAHATENSKIGVDVESIDSINISNETFIERILPASEQQYCQNAPSPQSSFAGRWSAKEAVFKSLGVCSKGAGAPLKDIEIENDSNGAPTLHGVAAEAAKEAGVKHISVSISHSDMQAVAVAISQF

SEQ ID NO: 21 - protein, 2091 aa, Yarrowia lipolytica
MYGTSTGPQTGINTPRSSQSLRPLILSHGSLEFSFLVPTSLHFHASQLKDTFTASLPEPTDELAQDDEPSSVAELVARYIGHVAHEVEEGEDDAHGTNQDVLKLTLNEFERAFMRGNDVHAVAATLPGITAKKVLVVEAYYAGRAAAGRPTKPYDSALFRAASDEKARIYSVLGGQGNIEEYFDELREVYNTYTSFVDDLISSSAELLQSLSREPDANKLYPKGLNVMQWLREPDTQPDVDYLVSAPVSLPLIGLVQLAHFAVTCRVLGKEPGEILERFSGTTGHSQGIVTAAAIATATTWESFHKAVANALTMLFWIGLRSQQAYPRTSIAPSVLQDSIENGEGTPTPMLSIRDLPRTAVQEHIDMTNQHLPEDRHISISLVNSARNFVVTGPPLSLYGLNLRLRKVKAPTGLDQNRVPFTQRKVRFVNRFLPITAPFHSQYLYSAFDRIMEDLEDVEISPKSLTIPVYGTKTGDDLRAISDANVVPALVRMITHDPVNWEQTTAFPNATHIVDFGPGGISGLGVLTNRNKDGTGVRVILAGSMDGTNAEVGYKPELFDRDEHSVKYAIDWVKEYGPRLVKNATGQTFVDTKMSRLLGIPPIMVAGMTPTTVPWDFVAATMNAGYHIELAGGGYYNAKTMTEAITKIEKAIPPGRGITVNLIYVNPRAMGWQIPLIGKLRADGVPIEGLTIGAGVPSIEVANEYIETLGIKHIAFKPGSVDAIQQVINIAKANPKFPVILQWTGGRGGGHHSFEDFHQPILQMYSRIRRHENIILVAGSGFGGAEDTYPYLSGNWSSRFGYPPMPFDGCLFGSRMMTAKEAHTSKNAKQAIVDAPGLDDQDWEKTYKGAAGGVVTVLSEMGEPIHKLATRGVLFWHEMDQKIFKLDKAKRVPELKKQRDYIIKKLNDDFQKVWFGRNSAGETVDLEDMTYAEVVHRMVDLMYVKHEGRWIDDSLKKLTGDFIRRVEERFTTAEGQASLLQNYSELNVPYPAVDNILAAYPEAATQLINAQDVQHFLLLCQRRGQKPVPFVPSLDENFEYWFKKDSLWQSEDLEAVVGQDVGRTCILQGPMAAKFSTVIDEPVGDILNSIHQGHIKSLIKDMYNGDETTIPITEYFGGRLSEAQEDIEMDGLTISEDANKISYRLSSSAADLPEVNRWCRLLAGRSYSWRHALFSADVFVQGHRFQTNPLKRVLAPSTGMYVEIANPEDAPKTVISVREPYQSGKLVKTVDIKLNEKGPIALTLYEGRTAENGVVPLTFLFTYHPDTGYAPIREVMDSRNDRIKEFYYRIWFGNKDVPFYTPTTATFNGGRETITSQAVADFVHAVGNTGEAFVERPGKEVFAPMDFAIVAGWKAITKPIFPRTIDGDLLKLVHLSNGFKMVPGAQPLKVGDVLDTTAQINSIINEESGKIVEVCGTIRRDGKPIMHVTSQFLYRGAYTDFENTFQRKDEVPMQVHLASSRDVAILRSKEWFRLDMDDVELLGQTLTFRLQSLIRFKNKNVFSQVQTMGQVLLELPTKEVIQVASVDYEAGTSHGNPVIDYLQRNGTSIEQPVYFENPIPLSGKTPLVLRAPASNETYARVSGDYNPIHVSRVFSSYANLPGTITHGMYTSAAVRSLVETWAAENNIGRVRGFHVSLVDMVLPNDLITVRLQHVGMIAGRKIIKVEASNKETEDKVLLGEAEVEQPVTAYVFTGQGSQEQGMGMELYATSPVAKEVWDRPSFHWNYGLSIIDIVKNNPKERTVHFGGPRGKAIRQNYMSMTFETVNADGTIKSEKIFKEIDETTTSYTYRSPTGLLSATQFTQPALTLMEKASFEDMRSKGLVQRDSSFAGHSLGEYSALADLADVMLIESLVSVVFYRGLTMQVAVERDEQGRSNYSMCAVNPSRISKTFNEQALQYVVGNISEQTGWLLEIVNYNVANMQYVAAGDLRALDCLTNLLNYLKAQNIDIPALMQSMSLEDVKAHLVNIIHECVKQTEAKPKPINLERGFATIPLKGIDVPFHSTFLRSGVKPFRSFLIKKINKTTIDPSKLVGKYIPNVTARPFEITKEYFEDVYRLTNSPRIAHILANWEKYEEGTEGGSRHGGTTAASS

SEQ ID NO: 22 - protein, 482 aa, Yarrowia lipolytica
MVKNVDQVDLSQVDTIASGRDVNYKVKYTSGVKMSQGAYDDKGRHISEQPFTWANWHQHINWLNFILVIALPLSSFAAAPFVSFNWKTAAFAVGYYMCTGLGITAGYHRMWAHRAYKAALPVRIILALFGGGAVEGSIRWWASSHRVHHRWTDSNKDPYDARKGFWFSHFGWMLLVPNPKNKGRTDISDLNNDWVVRLQHKYYVYVLVFMAIVLPTLVCGFGWGDWKGGLVYAGIMRYTFVQQVTFCVNSLAHWIGEQPFDDRRTPRDHALTALVTFGEGYHNFHHEFPSDYRNALIWYQYDPTKWLIWTLKQVGLAWDLQTFSQNAIEQGLVQQRQKKLDKWRNNLNWGIPIEQLPVIEFEEFQEQAKTRDLVLISGIVHDVSAFVEHHPGGKALIMSAVGKDGTAVFNGGVYRHSNAGHNLLATMRVSVIRGGMEVEVWKTAQNEKKDQNIVSDESGNRIHRAGLQATRVENPGMSGMAA

SEQ ID NO: 23 - protein, 2266 aa, Yarrowia lipolytica
MRLQLRTLTRRFFSMASGSSTPDVAPLVDPNIHKGLASHFFGLNSVHTAKPSKVKEFVASHGGHTVINKVLIANNGIAAVKEIRSVRKWAYETFGDERAISFTVMATPEDLAANADYIRMADQYVEVPGGTNNNNYANVELIVDVAERFGVDAVWAGWGHASENPLLPESLAASPRKIVFIGPPGAAMRSLGDKISSTIVAQHAKVPCIPWSGTGVDEVVVDKSTNLVSVSEEVYTKGCTTGPKQGLEKAKQIGFPVMIKASEGGGGKGIRKVEREEDFEAAYHQVEGEIPGSPIFIMQLAGNARHLEVQLLADQYGNNISLFGRDCSVQRRHQKIIEEAPVTVAGQQTFTAMEKAAVRLGKLVGYVSAGTVEYLYSHEDDKFYFLELNPRLQVEHPTTEMVTGVNLPAAQLQIAMGIPLDRIKDIRLFYGVNPHTTTPIDFDFSGEDADKTQRRPVPRGHTTACRITSEDPGEGFKPSGGTMHELNFRSSSNVWGYFSVGNQGGIHSFSDSQFGHIFAFGENRSASRKHMVVALKELSIRGDFRTTVEYLIKLLETPDFEDNTITTGWLDELISNKLTAERPDSFLAVVCGAATKAHRASEDSIATYMASLEKGQVPARDILKTLFPVDFIYEGQRYKFTATRSSEDSYTLFINGSRCDIGVRPLSDGGILCLVGGRSHNVYWKEEVGATRLSVDSKTCLLEVENDPTQLRSPSPGKLVKFLVENGDHVRANQPYAEIEVMKMYMTLTAQEDGIVQLMKQPGSTIEAGDILGILALDDPSKVKHAKPFEGQLPELGPPTLSGNKPHQRYEHCQNVLHNILLGFDNQVVMKSTLQEMVGLLRNPELPYLQWAHQVSSLHTRMSAKLDATLAGLIDKAKQRGGEFPAKQLLRALEKEASSGEVDALFQQTLAPLFDLAREYQDGLAIHELQVAAGLLQAYYDSEARFCGPNVRDEDVILKLREENRDSLRKVVMAQLSHSRVGAKNNLVLALLDEYKVADQAGTDSPASNVHVAKYLRPVLRKIVELESRASAKVSLKAREILIQCALPSLKERTDQLEHILRSSVVESRYGEVGLEHRTPRADILKEVVDSKYIVFDVLAQFFAHDDPWIVLAALELYIRRACKAYSILDINYHQDSDLPPVISWRFRLPTMSSALYNSVVSSGSKTPTSPSVSRADSVSDFSYTVERDSAPARTGAIVAVPHLDDLEDALTRVLENLPKRGAGLAISVGASNKSAAASARDAAAAAASSVDTGLSNICNVMIGRVDESDDDDTLIARISQVIEDFKEDFEACSLRRITFSFGNSRGTYPKYFTFRGPAYEEDPTIRHIEPALAFQLELARLSNFDIKPVHTDNRNIHVYEATGKNAASDKRFFTRGIVRPGRLRENIPTSEYLISEADRLMSDILDALEVIGTTNSDLNHIFINFSAVFALKPEEVEAAFGGFLERFGRRLWRLRVTGAEIRMMVSDPETGSAFPLRAMINNVSGYVVQSELYAEAKNDKGQWIFKSLGKPGSMHMRSINTPYPTKEWLQPKRYKAHLMGTTYCYDFPELFRQSIESDWKKYDGKAPDDLMTCNELILDEDSGELQEVNREPGANNVGMVAWKFEAKTPEYPRGRSFIVVANDITFQIGSFGPAEDQFFFKVTELARKLGIPRIYLSANSGARIGIADELVGKYKVAWNDETDPSKGFKYLYFTPESLATLKPDTVVTTEIEEEGPNGVEKRHVIDYIVGEKDGLGVECLRGSGLIAGATSRAYKDIFTLTLVTCRSVGIGAYLVRLGQRAIQIEGQPIILTGAPAINKLLGREVYSSNLQLGGTQIMYNNGVSHLTARDDLNGVHKIMQWLSYIPASRGLPVPVLPHKTDVWDRDVTFQPVRGEQYDVRWLISGRTLEDGAFESGLFDKDSFQETLSGWAKGVVVGRARLGGIPFGVIGVETATVDNTTPADPANPDSIEMSTSEAGQVWYPNSAFKTSQAINDFNHGEALPLMILANWRGFSGGQRDMYNEVLKYGSFIVDALVDYKQPIMVYIPPTGELRGGSWVVVDPTINSDMMEMYADVESRGGVLEPEGMVGIKYRRDKLLDTMARLDPEYSSLKKQLEESPDSEELKVKLSVREKSLMPIYQQISVQFADLHDRAGRMEAKGVIREALVWKDARRFFFWRIRRRLVEEYLITKINSILPSCTRLECLARIKSWKPATLDQGSDRGVAEWFDENSDAVSARLSELKKDASAQSFASQLRKDRQGTLQGMKQALASLSEAERAELLKGL

SEQ ID NO: 24 - protein, 514 aa, Yarrowia lipolytica
MTIDSQYYKSRDKNDTAPKIAGIRYAPLSTPLLNRCETFSLVWHIFSIPTFLTIFMLCCAIPLLWPFVIAYVVYAVKDDSPSNGGVVKRYSPISRNFFIWKLFGRYFPITLHKTVDLEPTHTYYPLDVQEYHLIAERYWPQNKYLRAIISTIEYFLPAFMKRSLSINEQEQPAERDPLLSPVSPSSPGSQPDKWINHDSRYSRGESSGSNGHASGSELNGNGNNGTTNRRPLSSASAGSTASDSTLLNGSLNSYANQIIGENDPQLSPTKLKPTGRKYIFGYHPHGIIGMGAFGGIATEGAGWSKLFPGIPVSLMTLTNNFRVPLYREYLMSLGVASVSKKSCKALLKRNQSICIVVGGAQESLLARPGVMDLVLLKRKGFVRLGMEVGNVALVPIMAFGENDLYDQVSNDKSSKLYRFQQFVKNFLGFTLPLMHARGVFNYDVGLVPYRRPVNIVVGSPIDLPYLPHPTDEEVSEYHDRYIAELQRIYNEHKDEYFIDWTEEGKGAPEFRMIE

(19) United States
(12) Patent Application Publication
(10) Pub. No.: US 2024/0279694 A1
(43) Pub. Date: Aug. 22, 2024

(54) PRODUCTION OF CANNABINOIDS IN YEAST

(71) Applicant: BIOMEDICAN, INC., Fremont, CA (US)

(72) Inventors: Maxim Mikheev, Fremont, CA (US); Difeng Gao, Union City, CA (US)

(21) Appl. No.: 18/582,231

(22) Filed: Feb. 20, 2024

Related U.S. Application Data
(62) Division of application No. 17/474,708, filed on Sep. 14, 2021, now Pat. No. 11,939,613.
Application No. 17/474,708 is a division of application No. 16/630,051, filed on Jan. 10, 2020, now Pat. No. 11,149,291.
(60) Provisional application No. 62/531,827, filed on Jul. 12, 2017.

(51) Int. Cl. C12P 17/06; C12N 9/00; C12N 9/02; C12N 9/04; C12N 9/10; C12N 9/88

(52) U.S. Cl. CPC C12P 17/06; C12N 9/0004; C12N 9/0006; C12N 9/1029; C12N 9/1085; C12N 9/88; C12N 9/93; C12Y 101/01034; C12Y 121/03007; C12Y 121/03008; C12Y 203/0102; C12Y 203/01206; C12Y 404/01026; C12Y 604/01002

(57) ABSTRACT

The present disclosure relates to the production of cannabinoids in yeast. In as aspect there is provided a genetically modified yeast comprising: one or more GPP producing genes and optionally, one or more GPP pathway genes; two or more olivetolic acid producing genes; one or more cannabinoid precursor or cannabinoid producing genes; one or more Hexanoyl-CoA producing genes, and at least 5% dry weight of fatty acids or fats.

### PRIORITY DOCUMENTS

The present application claims priority from U.S. Provisional Patent Application No. 62/531,827 titled “Production of terpenes including cannabinoids in yeasts” and filed on 12 Jul. 2017, the content of which is hereby incorporated by reference in its entirety.

### TECHNICAL FIELD

The present disclosure relates to the production of cannabinoids in yeast.

### BACKGROUND

Cannabinoids are a general class of chemicals that act on cannabinoid receptors and other target molecules to modulate a wide range of physiological behaviour such as neurotransmitter release. Cannabinoids are produced naturally in humans (called endocannabinoids) and by several plant species (called phytocannabinoids) including Cannabis sativa. Cannabinoids have been shown to have several beneficial medical/therapeutic effects and therefore they are an active area of investigation by the pharmaceutical industry for use as pharmaceutical products for various diseases.

Currently the production of cannabinoids for pharmaceutical or other use is done by chemical synthesis or through the extraction of cannabinoids from plants that are producing these cannabinoids, for example C. sativa. There are several drawbacks to the current methods of cannabinoid production. The chemical synthesis of various cannabinoids is a costly process when compared to the extraction of cannabinoids from naturally occurring plants. The chemical synthesis of cannabinoids also involves the use of chemicals that are not environmentally friendly, which can be considered as an additional cost to their production. Furthermore, the synthetic chemical production of various cannabinoids has been classified as less pharmacologically active as those extracted from plants such as C. sativa. Although there are drawbacks to chemically synthesized cannabinoids, the benefit of this production method is that the end product is a highly pure single cannabinoid. This level of purity is preferred for pharmaceutical use. The level of purity required by the pharmaceutical industry is reflected by the fact that no plant extract based cannabinoid production has received FDA approval yet and only synthetic compounds have been approved.

In contrast to the synthetic chemical production of cannabinoids, the other method that is currently used to produce cannabinoids is production of cannabinoids in plants that naturally produce these chemicals; the most used plant for this is C. sativa. In this method, the plant C. sativa is cultivated and during the flowering cycle various cannabinoids are produced naturally by the plant. The plant can be harvested and the cannabinoids can be ingested for pharmaceutical purposes in various methods directly from the plant itself or the cannabinoids can be extracted from the plant. There are multiple methods to extract the cannabinoids from the plant C. sativa. All of these methods typically involve placing the plant, C. sativa that contains the cannabinoids, into a chemical solution that selectively solubilizes the cannabinoids into this solution. There are various chemical solutions used to do this such as hexane, cold water extraction methods, CO2 extraction methods, and others. This chemical solution, now containing all the different cannabinoids, can then be removed, leaving behind the excess plant material. The cannabinoid containing solution can then be further processed for use.

There are several drawbacks of the natural production and extraction of cannabinoids in plants such as C. sativa. Since there are numerous cannabinoids produced by C. sativa it is often difficult to reproduce identical cannabinoid profiles in plants using an extraction process. Furthermore, variations in plant growth will lead to different levels of cannabinoids in the plant itself making reproducible extraction difficult. Different cannabinoid profiles will have different pharmaceutical effects which are not desired for a pharmaceutical product. Furthermore, the extraction of cannabinoids from C. sativa extracts produces a mixture of cannabinoids and not a highly pure single pharmaceutical compound. Since many cannabinoids are similar in structure it is difficult to purify these mixtures to a high level resulting in cannabinoid contamination of the end product.

There is thus a need to provide an improved method of cannabinoid production.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

According to a first aspect of the present disclosure, there is provided a genetically modified yeast comprising:
(a) one or more GPP producing genes and optionally, one or more GPP pathway genes;
(b) two or more olivetolic acid producing genes;
(c) one or more cannabinoid precursor or cannabinoid producing genes;
(d) one or more Hexanoyl-CoA producing genes, and
(e) at least 5% dry weight of fatty acids or fats.

In certain embodiments, the one or more GPP producing gene comprises at least one of:
a) a mutated farnesyl diphosphate synthase;
b) a mutated S. cerevisiae ERG20 comprising a K197E substitution;
c) a double mutated S. cerevisiae ERG20 comprising F96W and N127W substitutions;
d) a mutated Y. lipolytica ERG20 comprising a K189E substitution;
e) a double mutated Y. lipolytica ERG20 comprising F88W and N119W substitutions;
f) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 1-4;
g) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 1-4; or
h) any combination of (a)-(g).

In certain embodiments, the one or more GPP producing genes is selected from a GPP pathway gene. In certain embodiments, the GPP pathway gene is selected from:
a) a hydroxymethylglutaryl-CoA reductase (HMGR);
b) a truncated hydroxymethylglutaryl-CoA reductase (tHMGR);
c) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 5-6;
d) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 5-6; or
e) any combination of (a)-(d)

In certain embodiments, the two or more olivetolic acid producing genes comprise: at least one of (a)-(d) and at least one of (e)-(h); wherein (a)-(d) comprise:
a) an olivetol synthase (OLS);
b) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 7;
c) a polypeptide that has at least 70%, 75%, 80%, 85%, 90%, 91%, 92%, 93%, 94%, 95%, 96%, 97%, 98%, 99% or 100% sequence identity to SEQ ID NO: 7; or
d) any combination of (a)-(c);
and wherein (e)-(h) comprise:
e) an olivetolic acid cyclase (OAC);
f) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 8;
g) a polypeptide that has at least 70%, 75%, 80%, 85%, 90%, 91%, 92%, 93%, 94%, 95%, 96%, 97%, 98%, 99% or 100% sequence identity to SEQ ID NO: 8; or
h) any combination of (e)-(g).

In certain embodiments, the one or more cannabinoid precursor or cannabinoid producing genes comprise at least one of:
a) a soluble aromatic prenyltransferase;
b) a cannabigerolic acid synthase (CBGAS);
c) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 9-12;
d) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 9-12; or
e) any combination of (a)-(e);
alone or in combination with at least one of:
f) a tetrahydrocannabinolic acid synthase (THCAS);
g) a cannabidiolic acid synthase (CBDAS);
h) a cannabichromenic acid synthase (CBCAS);
i) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 13-15;
j) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 13-15; or
k) any combination of (f)-(j).

In certain embodiments, the soluble aromatic prenyltransferase is NphB from Streptomyces sp. strain CL190.

In certain embodiments, the one or more Hexanoyl-CoA producing genes comprise at least one of:
a) a hexanoyl-CoA synthase;
b) HexA and HexB;
c) StcJ and Stck;
d) a mutated FAS1 and a mutated FAS2;
e) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 16 and a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 17;
f) a polypeptide that has at least 70%, 75%, 80%, 85%, 90%, 91%, 92%, 93%, 94%, 95%, 96%, 97%, 98%, 99% or 100% sequence identity to SEQ ID NO: 16 and a polypeptide that has at least 70%, 75%, 80%, 85%, 90%, 91%, 92%, 93%, 94%, 95%, 96%, 97%, 98%, 99% or 100% sequence identity to SEQ ID NO: 17;
g) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 18 and a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 19;
h) a polypeptide that has at least 70%, 75%, 80%, 85%, 90%, 91%, 92%, 93%, 94%, 95%, 96%, 97%, 98%, 99% or 100% sequence identity to SEQ ID NO: 18 and a polypeptide that has at least 70%, 75%, 80%, 85%, 90%, 91%, 92%, 93%, 94%, 95%, 96%, 97%, 98%, 99% or 100% sequence identity to SEQ ID NO: 19;
i) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 20 and a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 21;
j) a polypeptide that has at least 70%, 75%, 80%, 85%, 90%, 91%, 92%, 93%, 94%, 95%, 96%, 97%, 98%, 99% or 100% sequence identity to SEQ ID NO: 20 and a polypeptide that has at least 70%, 75%, 80%, 85%, 90%, 91%, 92%, 93%, 94%, 95%, 96%, 97%, 98%, 99% or 100% sequence identity to SEQ ID NO: 21; or
k) any combination of (a)-(j).

In certain embodiments, the mutated FAS1 and FAS2 genes comprise the genetic modifications selected from: FAS1 I306A and FAS2 G1250S; FAS1 I306A, M1251W and FAS2 G1250S; or FAS1 I306A, R1834K and FAS2 G1250S.

In certain embodiments, the yeast comprises at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% dry weight of fatty acids or fats.

In certain embodiments, the yeast is genetically modified to produce at least 5%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% dry weight of fatty acids or fats

In certain embodiments, the yeast further comprises genetic modifications that increase the production of fatty acids or fats. In certain embodiments, the genetic modifications that increase the production of fatty acids or fats comprise at least one of:
a) delta-9 stearoyl-CoA desaturase (SCD);
b) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 22;
c) a polypeptide that has at least 70%, 75%, 80%, 85%, 90%, 91%, 92%, 93%, 94%, 95%, 96%, 97%, 98%, 99% or 100% sequence identity to SEQ ID NO: 22;
d) Acetyl-CoA carboxylase (ACC1);
e) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 23;
f) a polypeptide that has at least 70%, 75%, 80%, 85%, 90%, 91%, 92%, 93%, 94%, 95%, 96%, 97%, 98%, 99% or 100% sequence identity to SEQ ID NO: 23
g) Diacylglyceride acyl-transferase (DGA1);
h) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 24;
i) a polypeptide that has at least 70%, 75%, 80%, 85%, 90%, 91%, 92%, 93%, 94%, 95%, 96%, 97%, 98%, 99% or 100% sequence identity to SEQ ID NO: 24; or
j) any combination of (a)-(i).

In certain embodiments, the yeast is oleaginous. In certain embodiments, the yeast is selected from the genera Rhodosporidium, Rhodotorula, Yarrowia, Cryptococcus, Candida, Lipomyces and Trichosporon. The yeast of any one of the preceding claims, wherein the yeast is a Yarrowia lipolytica, a Lipomyces starkey, a Rhodosporidium toruloides, a Rhodotorula glutinis, a Trichosporon fermentans or a Cryptococcus curvatus.

According to a second aspect of the present disclosure, there is provided a method of producing at least one cannabinoid or cannabinoid precursor comprising contacting the yeast of the disclosure with a carbohydrate source under culture conditions and for a time sufficient to produce the at least one cannabinoid or cannabinoid precursor.

In certain embodiments, the at least one cannabinoid or cannabinoid precursor comprises CBGA, THCA, CBDA or CBCA.

According to a second aspect of the present disclosure, there is provided a cannabinoid precursor, cannabinoid or a combination thereof produced using the method of claim 17 or 18.

### BRIEF DESCRIPTION OF DRAWINGS

Embodiments of the present disclosure will be discussed with reference to the accompanying drawings wherein:

FIG. 1. is diagram of a cannabinoid synthesis pathway;

FIG. 2. is diagram of a cannabinoid synthesis pathway including nonenzymatic steps;

FIG. 3. is a high level scheme of yeast cell organisation; and

FIG. 4. Y. lipolytica oil content before and after modification. The pale circular regions indicate oils on the picture (see arrows). The ad9 mutant (right image) accumulates more oil than wild type Yarrowia lipolytica (left image).

### DESCRIPTION OF EMBODIMENTS

### Definitions

The following definitions are provided for specific terms which are used in the following written description.

As used in the specification and claims, the singular form “a”, “an” and “the” include plural references unless the context clearly dictates otherwise. For example, the term “a cannabinoid precursor” includes a plurality of precursors, including mixtures thereof. The term “a polynucleotide” includes a plurality of polynucleotides.

As used herein, the term “comprising” is intended to mean that the compositions and methods include the recited elements, but do not exclude other elements. “Consisting essentially of” shall mean excluding other elements of any essential significance to the combination. Thus, compositions consisting essentially of produced cannabinoids would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for produced cannabinoids. Embodiments defined by each of these transition terms are within the scope of this invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term ‘about’ means within an acceptable error range for the particular value, such as +1-20%, preferably +1-10% and more preferably +1-5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

As used herein, the terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, antisense molecules, cDNA, recombinant polynucleotides, branched polynucleotides, aptamers, plasmids, vectors, isolated DNA of any sequence, isolated RNA of any sequence, nucleic acid probes, and primers. A nucleic acid molecule may also comprise modified nucleic acid molecules (e.g., comprising modified bases, sugars, and/or internucleotide linkers).

As used herein, the term “peptide” refers to a compound of two or more subunit amino acids, amino acid analogs, or peptidomimetics. The subunits may be linked by peptide bonds or by other bonds (e.g., as esters, ethers, and the like).

As used herein, the term “amino acid” refers to either natural and/or unnatural or synthetic amino acids, including glycine and both D or L optical isomers, and amino acid analogs and peptidomimetics. A peptide of three or more amino acids is commonly called an oligopeptide if the peptide chain is short. If the peptide chain is long (e.g., greater than about 10 amino acids), the peptide is commonly called a polypeptide or a protein. While the term “protein” encompasses the term “polypeptide”, a “polypeptide” may be a less than full-length protein.

As used herein, “expression” refers to the process by which polynucleotides are transcribed into mRNA and/or translated into peptides, polypeptides, or proteins. If the polynucleotide is derived from genomic DNA, expression may include splicing of the mRNA transcribed from the genomic DNA.

As used herein, “under transcriptional control” or “operably linked” refers to expression (e.g., transcription or translation) of a polynucleotide sequence which is controlled by an appropriate juxtaposition of an expression control element and a coding sequence. In one aspect, a DNA sequence is “operatively linked” to an expression control sequence when the expression control sequence controls and regulates the transcription of that DNA sequence.

As used herein, “coding sequence” is a sequence which is transcribed and translated into a polypeptide when placed under the control of appropriate expression control sequences. The boundaries of a coding sequence are determined by a start codon at the 5′ (amino) terminus and a translation stop codon at the 3′ (carboxyl) terminus. A coding sequence can include, but is not limited to, a prokaryotic sequence, cDNA from eukaryotic mRNA, a genomic DNA sequence from eukaryotic (e.g., yeast, or mammalian) DNA, and even synthetic DNA sequences. A polyadenylation signal and transcription termination sequence will usually be located 3′ to the coding sequence.

As used herein, two coding sequences “correspond” to each other if the sequences or their complementary sequences encode the same amino acid sequences.

As used herein, “signal sequence” denotes the endoplasmic reticulum translocation sequence. This sequence encodes a signal peptide that communicates to a cell to direct a polypeptide to which it is linked (e.g., via a chemical bond) to an endoplasmic reticulum vesicular compartment, to enter an exocytic/endocytic organelle, to be delivered either to a cellular vesicular compartment, the cell surface or to secrete the polypeptide. This signal sequence is sometimes clipped off by the cell in the maturation of a protein. Signal sequences can be found associated with a variety of proteins native to prokaryotes and eukaryotes.

As used herein, “hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

As used herein, a polynucleotide or polynucleotide region (or a polypeptide or polypeptide region) which has a certain percentage (for example, at least about 50%, at least about 60%, at least about 70%, at least about 80%, at least about 85%, at least about 90%, at least about 95%, at least about 99%) of “sequence identity” to another sequence means that, when maximally aligned, using software programs routine in the art, that percentage of bases (or amino acids) are the same in comparing the two sequences.

Two sequences are “substantially homologous” or “substantially similar” when at least about 50%, at least about 60%, at least about 70%, at least about 75%, and preferably at least about 80%, and most preferably at least about 90 or 95% of the nucleotides match over the defined length of the DNA sequences. Similarly, two polypeptide sequences are “substantially homologous” or “substantially similar” when at least about 50%, at least about 60%, at least about 66%, at least about 70%, at least about 75%, and preferably at least about 80%, and most preferably at least about 90 or 95% of the amino acid residues of the polypeptide match over a defined length of the polypeptide sequence. Sequences that are substantially homologous can be identified by comparing the sequences using standard software available in sequence data banks. Substantially homologous nucleic acid sequences also can be identified in a Southern hybridization experiment under, for example, stringent conditions as defined for that particular system. Defining appropriate hybridization conditions is within the skill of the art. For example, stringent conditions can be: hybridization at 5×SSC and 50% formamide at 42° C., and washing at 0.1×SSC and 0.1% sodium dodecyl sulfate at 60° C. Further examples of stringent hybridization conditions include: incubation temperatures of about 25 degrees C. to about 37 degrees C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40 degrees C. to about 50 degrees C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 degrees C. to about 68 degrees C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Similarity can be verified by sequencing, but preferably, is also or alternatively, verified by function (e.g., ability to traffic to an endosomal compartment, and the like), using assays suitable for the particular domain in question.

The terms “percent (%) sequence similarity”, “percent (%) sequence identity”, and the like, generally refer to the degree of identity or correspondence between different nucleotide sequences of nucleic acid molecules or amino acid sequences of polypeptides that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wisconsin), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al, J. Mol. Biol. 1990; 215: 403. BLAST nucleotide searches can be performed with the NBLAST program, score=100, wordlength=12, to obtain nucleotide sequences homologous to sequences of the invention. BLAST protein searches can be performed with the XBLAST program, score=50, wordlength=3, to obtain amino acid sequences homologous to protein sequences of the invention. To obtain gapped alignments for comparison purposes, Gapped BLAST can be utilized as described in Altschul et al, Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/ on the WorldWideWeb.

Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, MA; available at accelrys.com on the WorldWideWeb), using either a Blossum 62 matrix or a PAM250 matrix, a gap weight of 16, 14, 12, 10, 8, 6, or 4, and a length weight of 1, 2, 3, 4, 5, or 6. In yet another preferred embodiment, the percent identity between two nucleotide sequences is determined using the GAP program in the GCG software package using a NWSgapdna.CMP matrix, a gap weight of 40, 50, 60, 70, or 80, and a length weight of 1, 2, 3, 4, 5, or 6. A particularly preferred set of parameters (and the one that can be used if the practitioner is uncertain about what parameters should be applied to determine if a molecule is a sequence identity or homology limitation of the invention) is using a Blossum 62 scoring matrix with a gap open penalty of 12, a gap extend penalty of 4, and a frameshift gap penalty of 5.

Another non-limiting example of how percent identity can be determined is by using software programs such as those described in Current Protocols In Molecular Biology (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

Statistical analysis of the properties described herein may be carried out by standard tests, for example, t-tests, ANOVA, or Chi squared tests. Typically, statistical significance will be measured to a level of p=0.05 (5%), more preferably p=0.01, p=0.001, p=0.0001, p=0.000001

“Conservatively modified variants” of domain sequences also can be provided. With respect to particular nucleic acid sequences, conservatively modified variants refer to those nucleic acids which encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues (Batzer, et al., 1991, Nucleic Acid Res. 19: 5081; Ohtsuka, et al., 1985, J. Biol. Chem. 260: 2605-2608; Rossolini et al., 1994, Mol. Cell. Probes 8: 91-98).

The term “biologically active fragment”, “biologically active form”, “biologically active equivalent” of and “functional derivative” of a wild-type protein, possesses a biological activity that is at least substantially equal (e.g., not significantly different from) the biological activity of the wild type protein as measured using an assay suitable for detecting the activity.

As used herein, the term “isolated” or “purified” means separated (or substantially free) from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. By substantially free or substantially purified, it is meant at least 50% of the population, preferably at least 70%, more preferably at least 80%, and even more preferably at least 90%, are free of the components with which they are associated in nature.

A cell has been “transformed”, “transduced”, or “transfected” when nucleic acids have been introduced inside the cell. Transforming DNA may or may not be integrated (covalently linked) with chromosomal DNA making up the genome of the cell. For example, the polynucleotide may be maintained on an episomal element, such as a plasmid or a stably transformed cell is one in which the polynucleotide has become integrated into a chromosome so that it is inherited by daughter cells through chromosome replication. This stability is demonstrated by the ability of the cell to establish cell lines or clones comprised of a population of daughter cells containing the transformed polynucleotide. A “clone” is a population of cells derived from a single cell or common ancestor by mitosis. A “cell line” is a clone of a primary cell that is capable of stable growth in vitro for many generations (e.g., at least about 10).

A “vector” includes plasmids and viruses and any DNA or RNA molecule, whether self-replicating or not, which can be used to transform or transfect a cell.

As used herein, a “genetic modification” refers to any addition, deletion or disruption to a cell's normal nucleotides and/or additional of heterologous sequences into the yeast. Any method which can achieve the genetic modification are within the spirit and scope of this invention. Art recognized methods include viral mediated gene transfer, liposome mediated transfer, transformation, transfection and transduction

The practice of the present invention employs, unless otherwise indicated, conventional molecular biology, microbiology, and recombinant DNA techniques within the skill of the art. Such techniques are explained fully in the literature. See, e.g., Maniatis, Fritsch & Sambrook, In Molecular Cloning: A Laboratory Manual (1982); DNA Cloning: A Practical Approach, Volumes I and II (D. N. Glover, ed., 1985); Oligonucleotide Synthesis (M. J. Gait, ed., 1984); Nucleic Acid Hybridization (B. D. Hames & S. J. Higgins, eds., 1985); Transcription and Translation (B. D. Hames & S. I. Higgins, eds., 1984); Animal Cell Culture (R. I. Freshney, ed., 1986); Immobilized Cells and Enzymes (IRL Press, 1986); B. Perbal, A Practical Guide to Molecular Cloning (1984).

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

### Genetically Modified Yeast Strains

Disclosed herein are genetically modified yeasts comprising one or more genetic modifications that result in the production of at least one cannabinoid or cannabinoid precursor and methods for their creation. The disclosed yeast may produce various cannabinoids from a simple sugar source, for example, where the main carbon source available to the yeast is a sugar (glucose, galactose, fructose, sucrose, honey, molasses, raw sugar, etc.). Genetic engineering of the yeast involves inserting various genes that produce the appropriate enzymes and/or altering the natural metabolic pathway in the yeast to achieve the production of a desired compound. Through genetic engineering of yeast, these metabolic pathways can be introduced into these yeast and the same metabolic products that are produced in the plant C. sativa can be produced by the yeast. The benefit of this method is that once the yeast is engineered, the production of the cannabinoid is low cost and reliable, and only a specific cannabinoid is produced or a subset is produced, depending on the organism and the genetic manipulation. The purification of the cannabinoid is straightforward since there is only a single cannabinoid or a selected few cannabinoids present in the yeast. The process is a sustainable process which is more environmentally friendly than synthetic production.

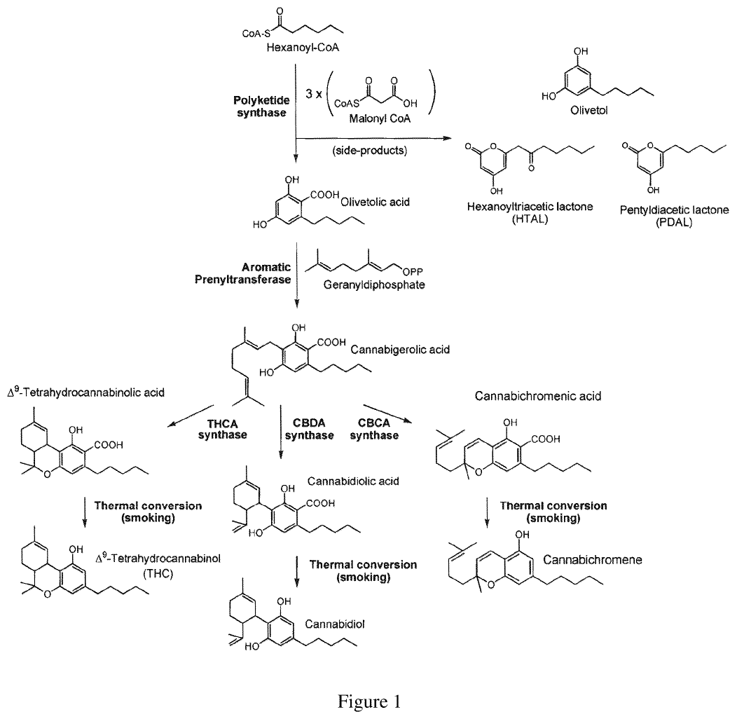

A high level biosynthetic route for the production of Cannabinoids is shown in FIG. 1. The pathway begins with the conversion of Hexanoyl-CoA to olivetolic acid (OA), a polyketide, by the action of polyketide synthase (OLS) and olivtolic acid cyclase (OAC). OA is then prenylated with the monoterpene geranyl diphosphate (GPP) to cannabigerolic acid (CBGA) by an aromatic prenyltransferase. Finally, cannabidiolic acid (CBDA) is produced by cyclizing CBGA via cannabidiolic acid synthase (CBDAS), or tettrahydrocannabinolic acid (THCA) is produced from CBGA by tetrahydrocannabinolic acid synthase (THCAS), or cannabichromenic acid (CBCA) is produced from CBGA by cannabichromenic acid synthase (CBCAS).

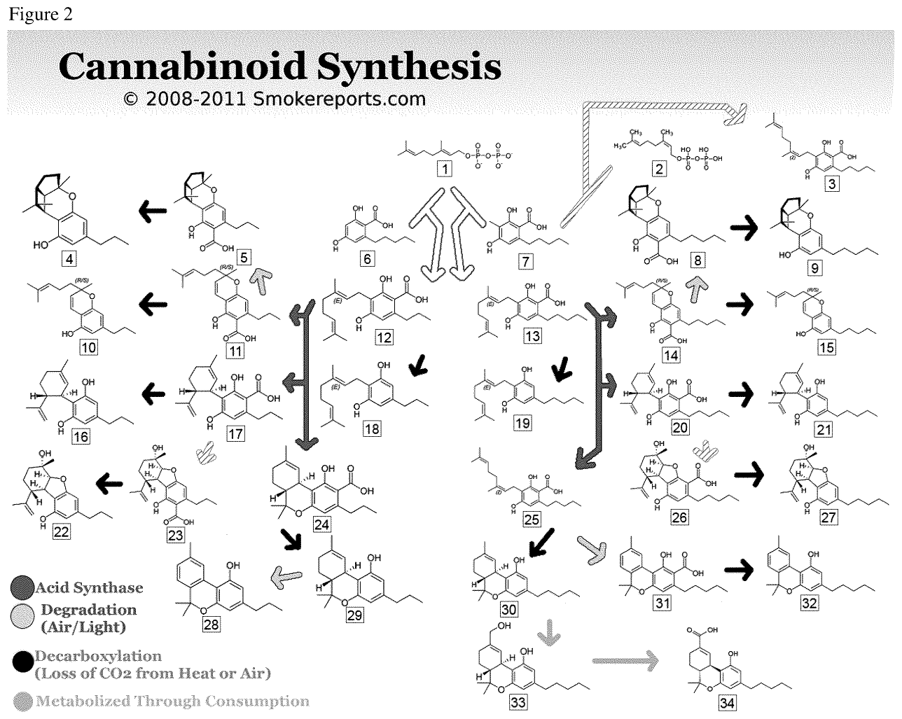

The production pathways for non-enzymatic modifications are demonstrated on FIG. 2.

In the past, there have been multiple attempts to produce cannabinoids in yeasts. At present, no one has been able reach a reasonable price for production due to extremely low yield. We have identified how the yield can be increased.

We have established a link between oil production in yeast and theoretical maximum cannabinoid production. Based on this unexpected link: (1) instead of producing cannabinoids in traditional yeasts, we propose using yeast having at least 5% dry weight of fatty acids or fats, such as oily yeasts, for example, Y. Lipolytica; (2) we also propose making additional genetic modifications that will increase oil production level in the engineered yeast; (3) add additional genes from the cannabinoid production pathway in combination with genes from alternative pathways that produce cannabinoid intermediates, such as for example NphB; (4) increase production of GPP by, for example, genetically mutating ERG20 and/or by using equivalent genes from alternative pathways; (5) increase production of compounds from fatty acid pathway for use in the cannabinoid production pathway, for example, increase the production of malonyl-CoA by overexpressing ACC1.

Cannabinoids have a limited solubility in water solutions. Yet, they have a high solubility in hydrophobic liquids like lipids, oils or fats. If hydrophobic media is limited or completely removed than CBGA will not be solubilized and will have limited availability to following cannabinoid synthetases. As an example, in the paper (Zirpel et al. 2015) it was shown that purified THCA synthase is almost unable to convert CBGA into THCA. In the same paper the authors demonstrated that unpurified yeast lysate converts CBGA much more efficiently. The authors also demonstrated that CBGA was dissolved in the lipid fraction. In another paper (Lange et al. 2016) the authors made the next step in improving a cell free process. They used a two-phase reaction with an organic, hydrophobic phase and aquatic phase. The authors demonstrated a high yield of THCA from CBGA. They found that CBGA was dissolved in organic phase. They also demonstrated that THCA was moved back to the organic phase. We can therefore conclude that a hydrophobic phase is required for successful synthesis and that cannabinoids are mostly present in the organic phase.

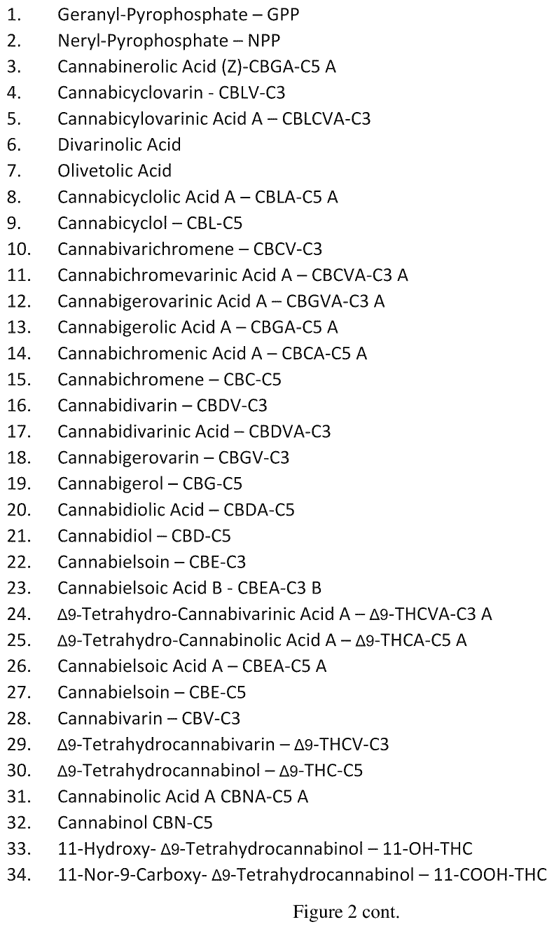

A high level scheme of a yeast cell is shown in FIG. 3. The main mass of lipids in traditional yeasts like S. cerevisiae, K. phaffii, K. marxianus are deposited in the lipid membrane. These types of yeast almost have no oily bodies. In such a case, any cannabinoids that are produced will be dissolved in this membrane. Too many cannabinoids will destabilize a membrane which will cause cell death. It was reported that in the best conditions, with high sugar content and without nitrogen supply, these yeasts can have a maximum of 2-3% dry weight of oils (ie fats and fatty acids).

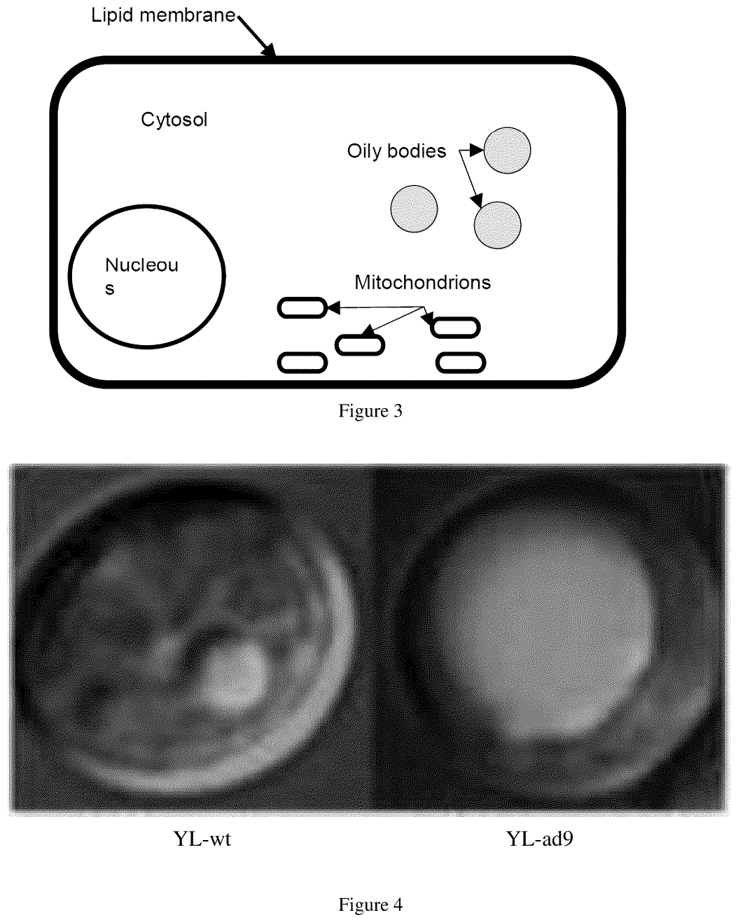

There are several non-traditional yeasts, like Y. lipolytica. The natural form of Y. lipolytica can have up to 17% dry weight of oils. The main mass of oil is located in oily bodies. Cannabinoids dissolved in such bodies will not cause membrane instability. As a result, Y. lipolytica can have a much higher cannabinoid production level. Several works have demonstrated modifications for Y. lipolytica which can bring the lipid content above 80% of dry mass (Qiao et al. 2015). FIG. 4. is taken from Qiao et al. 2015 and demonstrates the difference in oil content in modified vs wild type Y. lipolytica. A minimal set of 3 gene modifications were used.

Therefore, we propose that cannabinoids can be produced to some percentage of the oil content in yeast. This gives a correlation—more oil means more cannabinoid production.

A review paper (Ângela et al. 2017) analysed different types of yeast as a potential producers for cannabinoids. TABLE 1 is adapted from the summary table in Ângela et al. 2017, in which the authors compared 4 yeasts types by different parameters. Yet, they completely ignored oil content, theoretical maximal limit of production and minimal cost of goods for production. The far right two columns show maximum oil amount as a percentage of dry weight, and the production cost if there is only 1% of cannabinoid in the oil. The bottom row shows an embodiment of a modified Yarrowia lipolytica of the present disclosure. Finally, the authors in Ângela et al. 2017 considered that acetyl-CoA pool engineering had optimization potential; +. However, we have found that YL has large concentration of acetyl-CoA without modifications.

Therefore, we are proposing to use oily yeasts as a backbone for production.

TABLE 1
COMPARISON OF DIFFERENT MICROBIAL EXPRESSION HOSTS REGARDING
THEIR CAPACITY OF HETEROOGOUS CANNABINOID BIOSYNTHESIS

| | | | | | | | | | Production cost |
|---|---|---|---|---|---|---|---|---|---|
| | Genetic | Strains, | plant protein | Post- | | Hexanoic | acetyl-CoA | Maximal oil | with only 1% of |
| | tools | promoters, | expression | translational | GPP | acid | pool | amount % of | cannabinoids |
| | available | vectors | capacity | modifications | engineering | engineering | engineering | dry weight | from oils |
| E. coli | +++ | +++ | + | − | ++ | + | + | 2% | $12.50 |
| S. cerevisiae | +++ | +++ | ++ | ++ | +++ | ++ | +++ | 2% | $12.50 |
| P. Pastoris | + | ++ | +++ | ++ | + | ++ | | 3% | $8.33 |
| K. marxianus | ++ | + | ++ | ++ | | | | 3% | $8.33 |
| Y. Lipolica | + | + | ++ | ++ | + | ++ | +, YL has large | 17% | $1.47 |
| | | | | | | | concentration | | |
| | | | | | | | of ac-CoA | | |
| | | | | | | | without | | |
| | | | | | | | modifications | | |
| Y.L. | + | + | ++ | ++ | + | ++ | +, YL has large | 80% | $0.31 |
| modified | | | | | | | concentration | | |
| | | | | | | | of ac-CoA | | |
| | | | | | | | without | | |
| | | | | | | | modifications | | |

*maximal oil % means how much oils can be produced in a best cultivation conditions. % calculated from dried mass.
Table 1 adapted from Carvalho, Ângela, et al. “Designing microorganisms for heterologous biosynthesis of cannabinoids.” FEMS yeast research 17.4 (2017). 1. +++, many publications available, well established; ++, publications available, optimization potential; +, first publications available, not yet established/not working; “−”, not possible; ‘empty’, not yet described.

The pathway of cannabinoids is described in multiple publications. We can use all suggested modification from recent papers (Zirpel et al. 2017; Poulos and Farnia 2016; Ângela et al. 2017) and others. We can use modifications for a biosynthesis pathway like replacing CBGA synthase by NphB gene which already described (Zirpel et al. 2017).

As described in multiple papers for cannabinoid and terpenes production we also propose mutations that will increase production of GPP. For example, one way will use a mutated ERG20 gene to increase amount of GPP which is required in the cannabinoid synthesis pathway (Zhao et al. 2016; ZHUANG n.d.; Kampranis and Makris 2012).

There are a few key differences between plant polyketide and terpene biosynthesis when compared to yeast. Yeast does not contain many of the enzymes and fatty acids required for the production of THCA. Moreover, yeast do not express high levels of geranyl diphosphate (GPP), a chemical required for the production of cannabigerolic acid, the precursor to THCA. Yet, through genetic engineering many of the required enzymes can be added and the production of GPP can be increased. In order to add the required enzymes for cannabinoid production in yeast, the inventors have transformed yeast with the essential genes in the cannabinoid biosynthetic pathway.

Accordingly, in a first aspect, there is provided a genetically modified yeast comprising: (a) one or more GPP producing genes and optionally, one or more GPP pathway genes; (b) two or more olivetolic acid producing genes; (c) one or more cannabinoid precursor or cannabinoid producing genes; (d) one or more Hexanoyl-CoA producing genes, and (e) at least 5% dry weight of fatty acids or fats.

The disclosed genes may be endogenous or heterologous and include homologs that retain the function of the disclosed genes. As would be appreciated by the person skilled in the art, homologs could be identified through nucleotide or amino acid sequence alignments. In certain embodiments, the genetically modified yeast comprises: polynucleotides encoding polypeptides that are at least 70%, at least 75%, at least 80%, at least 85%, at least 90%, at least 95%, at least 96%, at least 97%, at least 98%, at least 99%, or 100% identical to the disclosed SEQ ID NOS; polypeptides that are at least 70%, at least 75%, at least 80% at least 85%, at least 90%, at least 95%, at least 96%, at least 97%, at least 98%, at least 99%, or 100% identical to the disclosed SEQ ID NOS; or both. Variants of the common cannabinoid synthesising proteins, such as CBDAS, retain the ability to cyclize CBGA to produce CBDA. For example, a variant common cannabinoid synthesising protein, such as CBDAS, must retain the ability to cyclize CBGA to produce CBDA with at least about 50%, at least about 60%, at least about 70%, at least about 80%, at least about 90%, or at least about 100% efficacy compared to the original sequence. In preferred embodiments, a variant common cannabinoid protein, such as CBDAS, has improved activity over the sequence from which it is derived in that the improved variant common cannabinoid protein has more than 110%, 120%, 130%, 140%, or and 150% improved activity in cyclizing CBGA to produce CBDA, as compared to the sequence from which the improved variant is derived.

The disclosed genes may be under the control of any suitable promoter. Many native promoters are available, for example, for Y. lipolytica, native promoters are available from the genes for translational elongation factor EF-1 alpha, acyl-CoA: diacylglycerol acyltransferase, acetyl-CoA-carboxylase 1, ATP citrate lyase 2, fatty acid synthase subunit beta, fatty acid synthase subunit alpha, isocitrate lyase 1, POX4 fatty-acyl coenzyme A oxidase, ZWF1 glucose-6-phosphate dehydrogenase, gytosolic NADP-specific isocitrate dehydrogenase, glyceraldehyde 3-phosphate dehydrogenase and the TEF intron (Wong et al. 2017). Any suitable terminator may be used. Short synthetic terminators are particularly suitable and are readily available, see for example, MacPherson et al. 2016.

As would be appreciated by the person skilled in the art, increased expression of a gene may provide increased the activity of the gene product. In certain embodiments, overexpression of a gene can increase the activity of the gene product in yeast by about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 95%, or about 200%.

As described above, in certain embodiments, the yeast comprises at least 5% dry weight of fatty acids or fats. Accordingly, the yeast may be oleaginous. Any oleaginous yeast may be suitable, however, particularly suitable yeast may be selected from the genera Rhodosporidium, Rhodotorula, Yarrowia, Cryptococcus, Candida, Lipomyces and Trichosporon. In certain embodiments, the yeast is a Yarrowia lipolytica, a Lipomyces starkey, a Rhodosporidium toruloides, a Rhodotorula glutinis, a Trichosporon fermentans or a Cryptococcus curvatus. The yeast may be naturally oleaginous. Accordingly, in certain embodiments, the yeast comprises at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 30%, at least 35%, at least 40%, at least 45%, at least 50%, at least 55%, at least 60%, at least 65%, at least 70%, at least 75% or at least 80% dry weight of fatty acids or fats. The yeast may also be genetically modified to accumulate or produce more fatty acids or fats. Accordingly, in certain embodiments, the yeast is genetically modified to produce at least 5%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 30%, at least 35%, at least 40%, at least 45%, at least 50%, at least 55%, at least 60%, at least 65%, at least 70%, at least 75% or at least 80% dry weight of fatty acids or fats.

### GPP Producing Genes

GPP may be produced in yeast by expressing mutated farnesyl diphosphate synthase. Normally in yeast, the farnesyl diphosphate synthase ERG20 condenses isopentenyl diphosphate (IPP) and dimethylallyl diphosphate (DMAP) to provide geranyl pyrophosphate (GPP) and then condenses two molecules of GPP to provide feranyl pyrophosphate (FPP). However, only a low level of GPP remains as ERG20 converts most of the GPP to FPP. More GPP is required for the commercial scale production of cannabinoids. Accordingly, mutated ERG20 that has a reduced or inability to produce FPP, may be used to increase the production of GPP. Two sets of mutations have been identified in S. cerevisiae that increase GPP production. The first mutation is a substitution of K197E and the second is a double substitution of F96W and N127W. As would be readily appreciated by the person skilled in the art, due to the high homology between ERG20 from S. cerevisiae and ERG20 from Y. lypolytica, equivalent mutations may be introduced into ERG20 from Y. lipolytica. In Y. lipolytica the first mutation is a substitution of K189E and the second is a double substitution of F88W and N119W. Introducing Y. lipolytica ERG20 (K189E) increases the production of GPP but growth is little bit slower compared to wild type yeast. Introducing Y. lipolytica ERG20 (F88W and N119W) produces fast growing clones with a high level of GPP. The sequences for the Y. lipolytica and S. cerevisiae genes are shown herein, however the skilled person would understand that homologous genes may also be suitable. Examples of ERG20 homologs as shown in Table 2. Accordingly, in certain embodiments, the one or more GPP producing genes comprise: a mutated farnesyl diphosphate synthase; a mutated S. cerevisiae ERG20 comprising a K197E substitution; a double mutated S. cerevisiae ERG20 comprising F96W and N127W substitutions; a mutated Y. lipolytica ERG20 comprising a K189E substitution; or a double mutated Y. lipolytica ERG20 comprising F88W and N119W substitutions; or a combination thereof. For the SEQ IDS described herein, mutations are shown with a solid underline. In certain embodiments, S. cerevisiae ERG20 (K197E) comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 1. In certain embodiments, S. cerevisiae ERG20 (K197E) comprises a polypeptide that has at least 70%, 75%, 80%, 85%, 90%, 91%, 92%, 93%, 94%, 95%, 96%, 97%, 98%, 99% or 100% sequence identity to SEQ ID NO: 1. In certain embodiments, S. cerevisiae ERG20 (F96W and N127W) comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 2. In certain embodiments, S. cerevisiae ERG20 (F96W and N127W) comprises a polypeptide that has at least 70%, 75%, 80%, 85%, 90%, 91%, 92%, 93%, 94%, 95%, 96%, 97%, 98%, 99% or 100% sequence identity to SEQ ID NO: 2. The equivalent Y. lipolytica amino acid sequences are shown in SEQ ID NOS: 3 and 4. In certain embodiments, Y. lipolytica ERG20 (K189E) comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 3. In certain embodiments, Y. lipolytica ERG20 (K189E) comprises a polypeptide that has at least 70%, 75%, 80%, 85%, 90%, 91%, 92%, 93%, 94%, 95%, 96%, 97%, 98%, 99% or 100% sequence identity to SEQ ID NO: 3. In certain embodiments, Y. lipolytica ERG20 (F88W and N119W) comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 4. In certain embodiments, Y. lipolytica ERG20 (F88W and N119W) comprises a polypeptide that has at least 70%, 75%, 80%, 85%, 90%, 91%, 92%, 93%, 94%, 95%, 96%, 97%, 98%, 99% or 100% sequence identity to SEQ ID NO: 4.

Variants of the GPP proteins, such as ERG20, retain the ability to, for example, condense isopentenyl diphosphate (IPP) and dimethylallyl diphosphate (DMAP) to geranyl pyrophosphate (GPP) and yet have reduced GPP to FPP activity. For example, a variant of a GPP protein, such as ERG20, retains the ability to condense isopentenyl diphosphate (IPP) and dimethylallyl diphosphate (DMAP) to geranyl pyrophosphate (GPP) with at least about at least about 80%, at least about 90%, or at least about 100% efficacy compared to the original sequence, while the ability to condense GPP to FPP is reduced by at least 10%, 20%, 30%, 40%, 50%, 60%, 70%, 80%, 90% or 100% (null mutation) as compared to the sequence from which it is derived.

ERG20 (K197E)
SEQ ID NO: 1
MASEKEIRRERFLNVFPKLVEELNASLLAYGMPKEACDWYAHSLNYNTPG
GKLNRGLSVVDTYAILSNKTVEQLGQEEYEKVAILGWCIELLQAYFLVAD
DMMDKSITRRGQPCWYKVPEVGEIAINDAFMLEAAIYKLLKSHFRNEKYY
IDITELFHEVTFQTELGQLMDLITAPEDKVDLSKFSLKKHSFIVTFETAY
YSFYLPVALAMYVAGITDEKDLKQARDVLIPLGEYFQIQDDYLDCFGTPE
QIGKIGTDIQDNKCSWVINKALELASAEQRKTLDENYGKKDSVAEAKCKK
IFNDLKIEQLYHEYEESIAKDLKAKISQVDESRGFKADVLTAFLNKVYKR
SK*
ERG20 (F96W and N127W)
SEQ ID NO: 2
MASEKEIRRERFLNVFPKLVEELNASLLAYGMPKEACDWYAHSLNYNTPG
GKLNRGLSVVDTYAILSNKTVEQLGQEEYEKVAILGWCIELLQAYWLVAD
DMMDKSITRRGQPCWYKVPEVGEIAIWDAFMLEAAIYKLLKSHFRNEKYY
IDITELFHEVTFQTELGQLMDLITAPEDKVDLSKFSLKKHSFIVTFKTAY
YSFYLPVALAMYVAGITDEKDLKQARDVLIPLGEYFQIQDDYLDCFGTPE
QIGKIGTDIQDNKCSWVINKALELASAEQRKTLDENYGKKDSVAEAKCKK
IFNDLKIEQLYHEYEESIAKDLKAKISQVDESRGFKADVLTAFLNKVYKR
SK*
Y. lipolytica ERG20 (K189E)
SEQ ID NO: 3
MSKAKFESVFPRISEELVQLLRDEGLPQDAVQWFSDSLQYNCVGGKLNRG
LSVVDTYQLLTGKKELDDEEYYRLALLGWLIELLQAFFLVSDDIMDESKT
RRGQPCWYLKPKVGMIAINDAFMLESGIYILLKKHFRQEKYYIDLVELFH
DISFKTELGQLVDLLTAPEDEVDLNRFSLDKHSFIVRYETAYYSFYLPVV
LAMYVAGITNPKDLQQAMDVLIPLGEYFQVQDDYLDNFGDPEFIGKIGTD
IQDNKCSWLVNKALQKATPEQRQILEDNYGVKDKSKELVIKKLYDDMKIE
QDYLDYEEEVVGDIKKKIEQVDESRGFKKEVLNAFLAKIYKRQK
Y. lipolytica ERG20 (F88W and N119W)
SEQ ID NO: 4
ASKAKFESVFPRISEELVQLLRDEGLPQDAVQWFSDSLQYNCVGGKLNRG
LSVVDTYQLLTGKKELDDEEYYRLALLGWLIELLQAFWLVSDDIMDESKT
RRGQPCWYLKPKVGMIAIWDAFMLESGIYILLKKHFRQEKYYIDLVELFH
DISFKTELGQLVDLLTAPEDEVDLNRFSLDKHSFIVRYKTAYYSFYLPVV
LAMYVAGITNPKDLQQAMDVLIPLGEYFQVQDDYLDNFGDPEFIGKIGTD
IQDNKCSWLVNKALQKATPEQRQILEDNYGVKDKSKELVIKKLYDDMKIE
QDYLDYEEEVVGDIKKKIEQVDESRGFKKEVLNAFLAKIYKRQK

TABLE 2
ERG20 HOMOLOGS

| Description | Ident | Accession |
|---|---|---|
| YALI0E05753p [Yarrowia lipolytica CLIB122] | 99% | XP_503599.1 |
| hypothetical protein [Nadsonia fulvescens var. elongata DSM 6958] | 71% | ODQ67901.1 |
| hypothetical protein [Lipomyces starkeyi NRRL Y-11557] | 70% | ODQ75043.1 |
| Farnesyl pyrophosphate synthetase [Galactomyces candidus] | 68% | CDO55796.1 |
| hypothetical protein [Kazachstania naganishii CBS 8797] | 68% | XP_022463460.1 |
| farnesyl pyrophosphate synthase [Saitoella complicata NRRL Y-17804] | 66% | XP_m9025287.1 |
| hypothetical protein [Tetrapisispora blattae CBS 6284] | 67% | XP_004179894.1 |
| hypothetical protein [Torulaspora delbrueckii] | 67% | XP_003680478.1 |
| unnamed protein product [Zymoseptoria tritici ST99CH_1E4] | 66% | SMR57088.1 |
| ERG20 farnesyl diphosphate synthase [Zymoseptoria tritici IPO323] | 66% | XP_003850094.1 |
| LAFE_0G04434g1_1 [Lachancea fermentati] | 68% | SCW03167.1 |
| ERG20-like protein [Saccharomyces kudriavzevii IFO 1802] | 66% | EJT43164.1 |
| hypothetical protein [Dactylellina haptotyla CBS 200.50] | 66% | EP537682.1 |
| CYFA0S07e04962g1_1 [Cyberlindnera fabianii] | 65% | CDR41679.1 |
| probable farnesyl pyrophosphate synthetase [Ramularia collo-cygni] | 65% | XP_023628194.1 |
| farnesyl pyrophosphate synthetase [Kluyveromyces marxianus DMKU3- | 65% | XP_022673909d |
| 1042] | | |
| polyprenyl synt-domain-containing protein [Sphaerulina musiva | 67% | XP_016759989.1 |
| SO2202] | | |

High levels of GPP production are dependent on adequate mevalonate production. Hydroxymethylglutaryl-CoA reductase (HMGR) catalyses the production of mevalonate from HMG-COA and NADPH. HMGR is a rate limiting step in the GPP pathway in yeast. Accordingly, overexpressing HMGR may increase flux through the pathway and increase the production of GPP. HMGR is a GPP pathway gene. Other GPP pathway genes include those genes that are involved in the GPP pathway, the products of which either directly produce GPP or produce intermediates in the GPP pathway, for example, ERG10, ERG13, ERG12, ERG8, ERG19, IDI1 or ERG20, The HMGR1 sequence from Y. lipolytica consists of 999 amino acids (aa) (SEQ ID NO: 5), of which the first 500 aa harbor multiple transmembrane domains and a response element for signal regulation. The remaining 499 C-terminal residues contain a catalytic domain and an NADPH-binding region. Truncated HMGR1 (tHmgR) has been generated by deleting the N-terminal 500 aa (Gao et al. 2017). tHMGR is able to avoid self-degradation mediated by its N-terminal domain and is thus stabilized in the cytoplasm, which increases flux through the GPP pathway. The N-terminal 500 aa are shown with a dashed underline in SEQ ID NO: 5. The N-terminal 500 aa are deleted in SEQ ID NO: 6. In certain embodiments, the one or more GPP pathway genes comprise a hydroxymethylglutaryl-CoA reductase (HMGR); a truncated hydroxymethylglutaryl-CoA reductase (tHMGR); or a combination thereof. The sequence for the Y. lipolytica gene are shown herein, however the skilled person would understand that homologous genes may also be suitable. Examples of HMGR homologs as shown in Table 3. In certain embodiments, HMGR comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 5. In certain embodiments, HMGR comprises a polypeptide that has at least 70%, 75%, 80%, 85%, 90%, 91%, 92%, 93%, 94%, 95%, 96%, 97%, 98%, 99% or 100% sequence identity to SEQ ID NO: 5. In certain embodiments, tHmgR comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 6. In certain embodiments, tHmgR comprises a polypeptide that has at least 70%, 75%, 80%, 85%, 90%, 91%, 92%, 93%, 94%, 95%, 96%, 97%, 98%, 99% or 100% sequence identity to SEQ ID NO: 6.

The GPP producing and GPP pathway genes may be expressed using, for example, a constitutive TEF intron promoter or native promoter (Wong et al. 2017) and synthesized short terminator (Curran et al. 2015). Increased production of GPP can be determined by overexpressing a single heterologous gene encoding linalool synthase and then determining the production of linalool using, for example, a colorimentric assay (Ghorai 2012). Increased production of GPP may be indicated by a linalool concentration of at least 0.5 mg/L, 0.7 mg/L, 0.9 mg/L or preferably at least about 1 mg/L.

HMGR1
SEQ ID NO: 5
MLQAAIGKIVGFAVNRPIHTVVLTSIVASTAYLAILDIAIPGFEGTQPISYYHPAAKSYDNPAD
WTHIAEADIPSDAYRLAFAQIRVSDVQGGEAPTIPGAVAVSDLDHRIVMDYKQWAPWTASNEQI
ASENHIWKHSFKDHVAFSWIKWFRWAYLRLSTLIQGADNFDIAVVALGYLAMHYTFFSLFRSMR
KVGSHFWLASMALVSSTFAFLLAVVASSSLGYRPSMITMSEGLPFLVVAIGFDRKVNLASEVLT
SKSSQLAPMVQVITKIASKALFEYSLEVAALFAGAYTGVPRLSQFCFLSAWILIFDYMFLLTFY
SAVLAIKFEINHIKRNRMIQDALKEDGVSAAVAEKVADSSPDAKLDRKSDVSLFGASGAIAVFK
IFMVLGFLGLNLINLTAIPHLGKAAAAAQSVTPITLSPELLHAIPASVPVVVTFVPSVVYEHSQ
LILQLEDALTTFLAACSKTIGDPVISKYIFLCLMVSTALNVYLFGATREVVRTQSVKVVEKHVP
IVIEKPSEKEEDTSSEDSIELTVGKQPKPVTETRSLDDLEAIMKAGKTKLLEDHEVVKLSLEGK
LPLYALEKQLGDNTRAVGIRRSIISQQSNTKTLETSKLPYLHYDYDRVFGACCENVIGYMPLPV
GVAGPMNIDGKNYHIPMATTEGCLVASTMRGCKAINAGGGVTTVLTQDGMTRGPCVSFPSLKRA
GAAKIWLDSEEGLKSMRKAFNSTSRFARLQSLHSTLAGNLLFIRFRTTTGDAMGMNMISKGVEH
SLAVMVKEYGFPDMDIVSVSGNYCTDKKPAAINWIEGRGKSVVAEATIPAHIVKSVLKSEVDAL
VELNISKNLIGSAMAGSVGGFNAHAANLVTAIYLATGQDPAQNVESSNCITLMSNVDGNLLISV
SMPSIEVGTIGGGTILEPQGAMLEMLGVRGPHIETPGANAQQLARIIASGVLAAELSLCSALAA
GHLVQSHMTHNRSQAPTPAKQSQADLQRLQNGSNICIRS
tHmgR
SEQ ID NO: 6
TQSVKVVEKHVPIVIEKPSEKEEDTSSEDSIELTVGKQPKPVTETRSLDDLEAIMKAGKTKLLE
DHEVVKLSLEGKLPLYALEKQLGDNTRAVGIRRSIISQQSNTKTLETSKLPYLHYDYDRVFGAC
CENVIGYMPLPVGVAGPMNIDGKNYHIPMATTEGCLVASTMRGCKAINAGGGVTTVLTQDGMTR
GPCVSFPSLKRAGAAKIWLDSEEGLKSMRKAFNSTSRFARLQSLHSTLAGNLLFIRFRTTTGDA
MGMNMISKGVEHSLAVMVKEYGFPDMDIVSVSGNYCTDKKPAAINWIEGRGKSVVAEATIPAHI
VKSVLKSEVDALVELNISKNLIGSAMAGSVGGFNAHAANLVTAIYLATGQDPAQNVESSNCITL
MSNVDGNLLISVSMPSIEVGTIGGGTILEPQGAMLEMLGVRGPHIETPGANAQQLARIIASGVL
AAELSLCSALAAGHLVQSHMTHNRSQAPTPAKQSQADLQRLQNGSNICIRS

TABLE 3
HMGR HOMOLOGS

| Description | Ident | Accession |
|---|---|---|
| YALI0E04807p [Yarrowia lipolytica CLIB122] | 100% | XP_503558.1 |
| hypothetical protein [Nadsonia fulvescens var. elongata DSM 6958] | 75% | ODQ651.59.1 |
| hypothtical protein [Galactomyces candidum] | 74% | CD055526.1 |
| hypothetical protein [Lipomyces starkeyi NRRL Y-11557] | 74% | ODQ70929.1 |
| hypothetical protein [Meyerozyma guilliermondii ATCC 6260] | 76% | EDK4061.4.2 |
| HMG1 [Sugiyamaella lignohabitans] | 73% | XP_018736018.1 |
| hypothetical protein [Meyerozyma guilliermondii ATCC 6260] | 76% | XP_001482757.1 |
| hypothetical protein [Babjeviella inositovora NRRL Y-12698] | 76% | XP_018984841.1 |
| DEHA2D09372p [Debaryomyces hansenii CBS767] | 75% | XP_458872.2 |
| 3-hydroxy-3-methylglutaryl-coenzyme A reductase 1 [[Candida] glabrata] | 75% | KTB22480.1 |
| hypothetical protein [Vanderwaltozyma polyspora DSM 70294] | 72% | XP_001643950.1 |
| LAFE oAm.552g1_1 [Lachancea fermentati] | 76% | SCV99364.1 |
| hypothetical protein [Debaryomyces fabryi] | 75% | XP_m5466829.1 |
| uncharacterized protein [Kuraishia capsulata CBS 1993] | 76% | XP_022457391.1 |
| uncharacterized protein [Candida] glabrata] | 75% | XP_449268.1 |

### Olivetolic Acid Producing Genes

The production of Olivetolic acid (OA) requires the substrates hexanoyl-CoA and 3 malonyl-CoA molecules, with the malonyl-CoA molecule produced in yeast naturally by ACC1 from Acetyl-CoA. Olivetolic acid production requires two enzymes for the condensation and subsequent cyclization of malonyl-CoA with hexanoyl-CoA. This process requires the tetraketide synthase, olivetolic synthase (OS), and the polyketide cyclase, olivetolic acid cyclase (OAC). In certain embodiments, the two or more olivetolic acid producing genes comprise olivetol synthase (OLS) and olivetolic acid cyclase (OAC). The sequences for the Cannabis sativa genes are shown herein, however the skilled person would understand that homologous genes may also be suitable. In certain embodiments, OLS comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 7. In certain embodiments, OLS comprises a polypeptide that has at least 70%, 75%, 80%, 85%, 90%, 91%, 92%, 93%, 94%, 95%, 96%, 97%, 98%, 99% or 100% sequence identity to SEQ ID NO: 7. In certain embodiments, OAC comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 8. In certain embodiments, OAC comprises a polypeptide that has at least 70%, 75%, 80%, 85%, 90%, 91%, 92%, 93%, 94%, 95%, 96%, 97%, 98%, 99% or 100% sequence identity to SEQ ID NO: 8.

Variants of the OA proteins, such as OAC, retain the ability to, for example, catalyze the C2-C7 aldol cyclization of linear pentyl tetra-β-ketide CoA as the substrate, to generate OA. For example, a variant of a OA protein, such as OAC, must retain the ability to catalyze the C2-C7 aldol cyclization of linear pentyl tetra-β-ketide CoA as the substrate, to generate OA with at least about 50%, at least about 60%, at least about 70%, at least about 80%, at least about 90%, or at least about 100% efficacy compared to the original sequence. In preferred embodiments, a variant of a OA protein, such as OAC, has improved activity over the sequence from which it is derived in that the improved variant common cannabinoid protein has more than 110%, 120%, 130%, 140%, or and 150% improved activity in catalyzing the C2-C7 aldol cyclization of linear pentyl tetra-β-ketide CoA as the substrate, to generate OA, as compared to the sequence from which the improved variant is derived.

The OA producing genes OLS and OAC may be expressed using, for example, a constitutive TEF intron promoter or native promoter (Wong et al. 2017) and synthesized short terminator (Curran et al. 2015). Increased production of OA may be determined using high-performance liquid chromatography (HPLC) or Liquid chromatography-mass spectrometry (LC/MS). As yeast do not produce OA endogenously, the presence of OA indicates that OLS and OAS are functioning.

OLS
SEQ ID NO: 7
MNHLRAEGPASVLAIGTANPENILLQDEFPDYYFRVTKSEHMTQLKEKFR
KICDKSMIRKRNCFLNEEHLKQNPRLVEHEMQTLDARQDMLVVEVPKLGK
DACAKAIKEWGQPKSKITELIFTSASTTDMPGADYHCAKLLGLSPSVKRV
MMYQLGCYGGGTVLRIAKDIAENNKGARVLAVCCDIMACLFRGPSESDLE
LLVGQAIFGDGAAAVIVGAEPDESVGERPIFELVSTGQTILPNSEGTIGG
HIREAGLIFDLHKDVPMLISNNIEKCLIEAFTPIGISDWNSIFWITHPGG
KAILDKVEEKLHLKSDKFVDSRHVLSEHGNMSSSTVLFVMDELRKRSLEE
GKSTTGDGFEWGVLFGFGPGLTVERVVVRSVPIKY
OAC
SEQ ID NO: 8
MAVKHLIVLKFKDEITEAQKEEFFKTYVNLVNIIPAMKDVYWGKDVTQKN
KEEGYTHIVEVTFESVETIQDYIIHPAHVGFGDVYRSFWEKLLIFDYTPR
K

### Cannabinoid Precursor or Cannabinoid Producing Genes

The production of the cannabinoids tetrahydrocannabinolic acid (THCA), cannabidiolic acid (CBDA) and cannabichromenic acid (CBCA) involves the prenylation of OA with GPP to CBGA by an aromatic prenyltransferase, and then CBDA, THCA or CBCA by CBDAS, THCAS or CBCAS, respectively.

Yeast do not naturally product CBGA, and therefore CBGA may be formed by heterologous expression of a CBGA synthase such as the membrane-bound CBGA synthase (CBGAS) from C. sativa. CBGAS is also known as geranylpyrophosphate olivetolate geranyltransferase, of which there are several forms, CsPT1, CsPT3 and CsPT4. In certain embodiments, the one or more cannabinoid precursor or cannabinoid producing genes comprise: a soluble aromatic prenyltransferase; a cannabigerolic acid synthase (CBGAS); or a combination thereof; either alone or in combination with the cannabinoid producing genes: tetrahydrocannabinolic acid synthase (THCAS); cannabidiolic acid synthase (CBDAS); cannabichromenic acid synthase (CBCAS); or any combination thereof. The sequences for the Cannabis sativa genes CBGAS, THCAS, CBDAS and CBCAS are shown herein, however the skilled person would understand that homologous genes may also be suitable.

In certain embodiments, CBGA synthase comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 9. In certain embodiments, CBGA synthase comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 10. In certain embodiments, CBGA synthase comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 11. In certain embodiments, CBGA synthase comprises a polypeptide that has at least 70%, 75%, 80%, 85%, 90%, 91%, 92%, 93%, 94%, 95%, 96%, 97%, 98%, 99% or 100% sequence identity to SEQ ID NOS: 9, 10 or 11. CBGA may also be formed by heterologous expression of a soluble aromatic prenyltransferase. In certain embodiments, the soluble aromatic prenyltransferase is NphB from Streptomyces sp. strain CL190 (ie wild type NphB) (Bonitz et al., 2011; Kuzuyama et al., 2005; Zirpel et al., 2017). In certain embodiments, the soluble aromatic prenyltransferase is NphB, comprising a mutation of a Q161A substitution. NphB (Q161A) produces more CBGA that wild type NphB (Muntendam 2015). Wild type NphB produces 15% CBGA and 85% of another by-product. The sequence for the Streptomyces sp. strain CL190 gene NphB is shown herein, however the skilled person would understand that homologous genes may also be suitable. In certain embodiments, NphB comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 12. In certain embodiments, NphB comprises a polypeptide that has at least 70%, 75%, 80%, 85%, 90%, 91%, 92%, 93%, 94%, 95%, 96%, 97%, 98%, 99% or 100% sequence identity to SEQ ID NO: 12.

Variants of the cannabinoid precursor or cannabinoid producing protein, such as NphB (Q161A), retains the ability to attach geranyl groups to aromatic substrates—such as converting OA and GPP to CBGA. For example, a variant Cannabinoid precursor or cannabinoid producing protein, such as NphB (Q161A), must retain the ability to attach geranyl groups to aromatic substrates, such as converting OA and GPP to CBGA, with at least about 50%, at least about 60%, at least about 70%, at least about 80%, at least about 90%, or at least about 100% efficacy compared to the original sequence. In preferred embodiments, a variant of a Cannabinoid precursor or cannabinoid producing protein, such as NphB (Q161A), has improved activity over the sequence from which it is derived in that the improved variant common cannabinoid protein has more than 110%, 120%, 130%, 140%, or and 150% improved activity in attach geranyl groups to aromatic substrates, such as converting OA and GPP to CBGA, as compared to the sequence from which the improved variant is derived.

The cannabinoid precursor or cannabinoid producing genes CBGAS, soluble aromatic prenyltransferase, CBGAS, THCAS, CBDAS and CBCAS may be expressed using, for example, a constitutive TEF intron promoter or native promoter (Wong et al. 2017) and synthesized short terminator (Curran et al. 2015). The production of one or more cannabinoid precursors or cannabinoids may be determined using a variety of methods. For example, if all of the precursors are available in the yeast cell, then the presence of the product, such as THCA, may be determined using HPLC or gas chromatography (GC). Alternatively, if only a portion of the cannabinoid synthesis pathway present, then cannabinoids will not be present and the activity of one or more genes can be checked by adding a gene and precursor. For example, to check CBGAS activity, OA and GPP are added to a crude cellular lysate. For checking CBCAS, THCAS or CBDAS activity, CBGA is added to a crude cellular lysate. A crude lysate or purified proteins may be used. Further, it may be necessary to use an aqueous/organic two-liquid phase setup in order to solubilize the hydrophobic substrate (eg CBGA) and to allow in situ product removal.

CsPT1
SEQ ID NO: 9
MGLSSVCTFSFQTNYHTLLNPHNNNPKTSLLCYRHPKTPIKYSYNNFPSKHCSTKSFHLQNKCS
ESLSIAKNSIRAATTNQTEPPESDNHSVATKILNFGKACWKLQRPYTIIAFTSCACGLFGKELL
HNTNLISWSLMFKAFFFLVAILCIASFTTTINQIYDLHIDRINKPDLPLASGEISVNTAWIMSI
IVALFGLIITIKMKGGPLYIFGYCFGIFGGIVYSVPPFRWKQNPSTAFLLNFLAHIITNFTFYY
ASRAALGLPFELRPSFTFLLAFMKSMGSALALIKDASDVEGDTKFGISTLASKYGSRNLTLFCS
GIVLLSYVAAILAGIIWPQAFNSNVMLLSHAILAFWLILQTRDFALTNYDPEAGRRFYEFMWKL
YYAEYLVYVFI
CsPT3
SEQ ID NO: 10
MGLSLVCTFSFQTNYHTLLNPHNKNPKNSLLSYQHPKTPIIKSSYDNFPSKYCLTKNFHLLGLN
SHNRISSQSRSIRAGSDQIEGSPHHESDNSIATKILNFGHTCWKLQRPYVVKGMISIACGLFGR
ELFNNRHLFSWGLMWKAFFALVPILSFNFFAAIMNQIYDVDIDRINKPDLPLVSGEMSIETAWI
LSIIVALTGLIVTIKLKSAPLFVFIYIFGIFAGFAYSVPPIRWKQYPFTNFLITISSHVGLAFT
SYSATTSALGLPFVWRPAFSFIIAFMTVMGMTIAFAKDISDIEGDAKYGVSTVATKLGARNMTF
VVSGVLLLNYLVSISIGIIWPQVFKSNIMILSHAILAFCLIFQTRELALANYASAPSRQFFEFI
WLLYYAEYFVYVFI
CsPT4
SEQ ID NO: 11
MVFSSVCSFPSSLGTNFKLVPRSNFKASSSHYHEINNFINNKPIKFSYFSSRLYCSAKPIVHRE
NKFTKSFSLSHLQRKSSIKAHGEIEADGSNGTSEFNVMKSGNAIWRFVRPYAAKGVLFNSAAMF
AKELVGNLNLFSWPLMFKILSFTLVILCIFVSTSGINQIYDLDIDRLNKPNLPVASGEISVELA
WLLTIVCTISGLTLTIITNSGPFFPFLYSASIFFGFLYSAPPFRWKKNPFTACFCNVMLYVGTS
VGVYYACKASLGLPANWSPAFCLLFWFISLLSIPISIAKDLSDIEGDRKFGIITFSTKFGAKPI
AYICHGLMLLNYVSVMAAAIIWPQFFNSSVILLSHAFMAIWVLYQAWILEKSNYATETCQKYYI
FLWIIFSLEHAFYLFM
NphB
SEQ ID NO: 12
MSEAADVERVYAAMEEAAGLLGVACARDKIYPLLSTFQDTLVEGGSVVVFSMASGRHSTELDFS
ISVPTSHGDPYATVVEKGLFPATGHPVDDLLADTQKHLPVSMFAIDGEVTGGFKKTYAFFPTDN
MPGVAELSAIPSMPPAVAENAELFARYGLDKVAMTSMDYKKRQVNLYFSELSAQTLEAESVLAL
VRELGLHVPNELGLKFCKRSFSVYPTLNWETGKIDRLCFAVISNDPTLVPSSDEGDIEKFHNYA
TKAPYAYVGEKRTLVYGLTLSPKEEYYKLGAYYHITDVQRGLLKAFDSLED

Producing CBGA is an initial step in producing many cannabinoids from C. sativa in yeast. Once CBGA is produced a single additional enzymatic step is required to turn CBGA into many other cannabinoids (CBDA, THCA, CBCA, etc.). The acidic forms of the cannabinoids can be used as a pharmaceutical product or the acidic cannabinoids can be turned into their neutral form for use, for example Cannabidiol (CBD) is produced from CBDA through decarboxylation. The resulting cannabinoid products will be used in the pharmaceutical/nutraceutical industry to treat a wide range of health issues. The genes for tetrahydrocannabinolic acid synthase (THCAS), cannabidiolic acid synthase (CBDAS) and cannabichromenic acid synthase (CBCAS) may be derived from C. sativa, however, the skilled person would understand that homologous genes may also be suitable. In certain embodiments, THCAS comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 13. In certain embodiments, THCAS comprises a polypeptide that has at least 70%, 75%, 80%, 85%, 90%, 91%, 92%, 93%, 94%, 95%, 96%, 97%, 98%, 99% or 100% sequence identity to SEQ ID NO: 13. In certain embodiments, CBDAS comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 14. In certain embodiments, CBDAS comprises a polypeptide that has at least 70%, 75%, 80%, 85%, 90%, 91%, 92%, 93%, 94%, 95%, 96%, 97%, 98%, 99% or 100% sequence identity to SEQ ID NO: 14. In certain embodiments, CBCAS comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 15. In certain embodiments, CBCAS comprises a polypeptide that has at least 70%, 75%, 80%, 85%, 90%, 91%, 92%, 93%, 94%, 95%, 96%, 97%, 98%, 99% or 100% sequence identity to SEQ ID NO: 15. Accordingly, in certain embodiments, the one or more cannabinoid precursor or cannabinoid producing genes comprise soluble aromatic prenyltransferase, cannabigerolic acid synthase (CBGAS), tetrahydrocannabinolic acid synthase (THCAS), cannabidiolic acid synthase (CBDAS) and cannabichromenic acid synthase (CBCAS).

THCAS
SEQ ID NO: 13
NPRENFLKCFSKHIPNNVANPKLVYTQHDQLYMSILNSTIQNLRFISDTTPKPLVIVTPSNNSH
IQATILCSKKVGLQIRTRSGGHDAEGMSYISQVPFVVVDLRNMHSIKIDVHSQTAWVEAGATLG
EVYYWINEKNENLSFPGGYCPTVGVGGHFSGGGYGALMRNYGLAADNIIDAHLVNVDGKVLDRK
SMGEDLFWAIRGGGGENFGIIAAWKIKLVAVPSKSTIFSVKKNMEIHGLVKLFNKWQNIAYKYD
KDLVLMTHFITKNITDNHGKNKTTVHGYFSSIFHGGVDSLVDLMNKSFPELGIKKTDCKEFSWI
DTTIFYSGVVNFNTANFKKEILLDRSAGKKTAFSIKLDYVKKPIPETAMVKILEKLYEEDVGAG
MYVLYPYGGIMEEISESAIPFPHRAGIMYELWYTASWEKQEDNEKHINWVRSVYNFTTPYVSQN
PRLAYLNYRDLDLGKTNHASPNNYTQARIWGEKYFGKNFNRLVKVKTKVDPNNFFRNEQSIPPL
PPHHH
CBDAS
SEQ ID NO: 14
NPRENFLKCFSQYIPNNATNLKLVYTQNNPLYMSVLNSTIHNLRFTSDTTPKPLVIVTPSHVSH
IQGTILCSKKVGLQIRTRSGGHDSEGMSYISQVPFVIVDLRNMRSIKIDVHSQTAWVEAGATLG
EVYYWVNEKNENLSLAAGYCPTVCAGGHFGGGGYGPLMRNYGLAADNIIDAHLVNVHGKVLDRK
SMGEDLFWALRGGGAESFGIIVAWKIRLVAVPKSTMFSVKKIMEIHELVKLVNKWQNIAYKYDK
DLLLMTHFITRNITDNQGKNKTAIHTYFSSVFLGGVDSLVDLMNKSFPELGIKKTDCRQLSWID
TIIFYSGVVNYDTDNFNKEILLDRSAGQNGAFKIKLDYVKKPIPESVFVQILEKLYEEDIGAGM
YALYPYGGIMDEISESAIPFPHRAGILYELWYICSWEKQEDNEKHLNWIRNIYNFMTPYVSKNP
RLAYLNYRDLDIGINDPKNPNNYTQARIWGEKYFGKNFDRLVKVKTLVDPNNFFRNEQSIPPLP
RHRH
CBCAS
SEQ ID NO: 15
NPQENFLKCFSEYIPNNPANPKFIYTQHDQLYMSVLNSTIQNLRFTSDTTPKPLVIVTPSNVSH
IQASILCSKKVGLQIRTRSGGHDAEGLSYISQVPFAIVDLRNMHTVKVDIHSQTAWVEAGATLG
EVYYWINEMNENFSFPGGYCPTVGVGGHFSGGGYGALMRNYGLAADNIIDAHLVNVDGKVLDRK
SMGEDLFWAIRGGGGENFGIIAAWKIKLVVVPSKATIFSVKKNMEIHGLVKLFNKWQNIAYKYD
KDLMLTTHFRTRNITDNHGKNKTTVHGYFSSIFLGGVDSLVDLMNKSFPELGIKKTDCKELSWI
DTTIFYSGVVNYNTANFKKEILLDRSAGKKTAFSIKLDYVKKLIPETAMVKILEKLYEEEVGVG
MYVLYPYGGIMDEISESAIPFPHRAGIMYELWYTATWEKQEDNEKHINWVRSVYNFTTPYVSQN
PRLAYLNYRDLDLGKTNPESPNNYTQARIWGEKYFGKNFNRLVKVKTKADPNNFFRNEQSIPPL
PPRHH

### Hexanoyl-CoA Producing Genes

A first step in the pathway for cannabinoid production in C. sativa begins with the conversion of hexanoic acid (a simple fatty acid) to hexanoyl-CoA by hexanoyl-CoA synthetase. For cannabinoid production in yeast, hexanoyl-CoA may be produced by expression of hexanoyl-CoA synthases HexA & HexB or StcJ & Stck, or mutated FAS1&2. Yeast do not naturally produce hexanoyl-CoA. The genes HexA & HexB encode the alpha (hexA) and beta (hexB) subunits of the hexanoate synthase (HexS) from Aspergillus parasiticus SU-1 (Hitchman et al. 2001). The genes StcJ and Stck are from Aspergillus nidulans and encode yeast-like FAS proteins (Brown et al. 1996). As would be understood by the person skilled in the art, many fungi would have hexanoate synthase or fatty acid synthase genes, which could readily be identified by sequencing of the DNA and sequence alignments with the known genes disclosed herein. Similarly, the skilled person would understand that homologous genes in different organisms may also be suitable. Examples of HexA and HexB homologs as shown in Tables 4 and 5. Examples of FAS1 and FAS2 homologs as shown in Tables 6 and 7. The endogenous yeast genes FAS1 (Fatty acid synthase subunit beta) and FAS2 (Fatty acid synthase subunit alpha) form fatty acid synthase FAS which catalyses the formation of long-chain fatty acids from acetyl-CoA, malonyl-CoA and NADPH. Mutated FAS produces short-chain fatty acids, such as hexanoic acid. Several different combinations of mutations enable the production of hexanoic acid. The mutations include: FAS1 I306A and FAS2 G1250S; FAS1 I306A and FAS2 G1250S and M1251W; and FAS1 I306A, R1834K and FAS2 G1250S (Gajewski et al. 2017). Mutated FAS2 and FAS1 may be expressed under the control of any suitable promoter, including, but not limited to the alcohol dehydrogenase II promoter of Y. lipolytica. Alternatively, genomic FAS2 and FAS1 can be directly mutated using, for example, homologous recombination or CRISPR-Cas9 genome editing technology.

Accordingly, in certain embodiments, HexA comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 16. In certain embodiments, HexA comprises a polypeptide that has at least 70%, 75%, 80%, 85%, 90%, 91%, 92%, 93%, 94%, 95%, 96%, 97%, 98%, 99% or 100% sequence identity to SEQ ID NO: 16. In certain embodiments, HexB comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 17. In certain embodiments, HexB comprises a polypeptide that has at least 70%, 75%, 80%, 85%, 90%, 91%, 92%, 93%, 94%, 95%, 96%, 97%, 98%, 99% or 100% sequence identity to SEQ ID NO: 17. In certain embodiments, StcJ comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 18. In certain embodiments, StcJ comprises a polypeptide that has at least 70%, 75%, 80%, 85%, 90%, 91%, 92%, 93%, 94%, 95%, 96%, 97%, 98%, 99% or 100% sequence identity to SEQ ID NO: 18. In certain embodiments, Stck comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 19. In certain embodiments, Stck comprises a polypeptide that has at least 70%, 75%, 80%, 85%, 90%, 91%, 92%, 93%, 94%, 95%, 96%, 97%, 98%, 99% or 100% sequence identity to SEQ ID NO: 19. In certain embodiments, FAS2 comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 20 and one of the combinations of mutations defined above. In certain embodiments, FAS2 comprises a polypeptide that has at least 70%, 75%, 80%, 85%, 90%, 91%, 92%, 93%, 94%, 95%, 96%, 97%, 98%, 99% or 100% sequence identity to SEQ ID NO: 20 and one of the combinations of mutations defined above. In certain embodiments, FAS1 comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 21 and one of the combinations of mutations defined above. In certain embodiments, FAS1 comprises a polypeptide that has at least 70%, 75%, 80%, 85%, 90%, 91%, 92%, 93%, 94%, 95%, 96%, 97%, 98%, 99% or 100% sequence identity to SEQ ID NO: 21 and one of the combinations of mutations defined above.

Variants of the Hexanoyl-CoA producing proteins retain the ability to catalyse the formation of long-chain fatty acids from acetyl-CoA, malonyl-CoA and NADPH. For example, a variant of a Hexanoyl-CoA producing protein must retain the ability to catalyse the formation of long-chain fatty acids from acetyl-CoA, malonyl-CoA and NADPH with at least about 50%, at least about 60%, at least about 70%, at least about 80%, at least about 90%, or at least about 100% efficacy compared to the original sequence. In preferred embodiments, a variant of a Hexanoyl-CoA producing protein has improved activity over the sequence from which it is derived in that the improved variant common cannabinoid protein has more than 110%, 120%, 130%, 140%, or and 150% improved activity in catalysing the formation of long-chain fatty acids from acetyl-CoA, malonyl-CoA and NADPH, as compared to the sequence from which the improved variant is derived.

The hexanoyl-CoA synthases HexA & HexB, StcJ & Stck, or mutated FAS1&2 may be expressed using, for example, a constitutive TEF intron promoter or native promoter (Wong et al. 2017) and synthesized short terminator (Curran et al. 2015). The production of hexanoyl-CoA in yeast may be determined by directly measuring the concentration of hexanoyl-CoA extracted from yeast cells using LC-MS.

HexA
SEQ ID NO: 16
MVIQGKRLAASSIQLLASSLDAKKLCYEYDERQAPGVTQITEEAPTEQPPLSTPPSLPQTPNIS
PISASKIVIDDVALSRVQIVQALVARKLKTAIAQLPTSKSIKELSGGRSSLQNELVGDIHNEFS
SIPDAPEQILLRDFGDANPTVQLGKTSSAAVAKLISSKMPSDFNANAIRAHLANKWGLGPLRQT
AVLLYAIASEPPSRLASSSAAEEYWDNVSSMYAESCGITLRPRQDTMNEDAMASSAIDPAVVAE
FSKGHRRLGVQQFQALAEYLQIDLSGSQASQSDALVAELQQKVDLWTAEMTPEFLAGISPMLDV
KKSRRYGSWWNMARQDVLAFYRRPSYSEFVDDALAFKVFLNRLCNRADEALLNMVRSLSCDAYF
KQGSLPGYHAASRLLEQAITSTVADCPKARLILPAVGPHTTITKDGTIEYAEAPRQGVSGPTAY
IQSLRQGASFIGLKSADVDTQSNLTDALLDAMCLALHNGISFVGKTFLVTGAGQGSIGAGVVRL
LLEGGARVLVTTSREPATTSRYFQQMYDNHGAKFSELRVVPCNLASAQDCEGLIRHVYDPRGLN
WDLDAILPFAAASDYSTEMHDIRGQSELGHRLMLVNVFRVLGHIVHCKRDAGVDCHPTQVLLPL
SPNHGIFGGDGMYPESKLALESLFHRIRSESWSDQLSICGVRIGWTRSTGLMTAHDIIAETVEE
HGIRTFSVAEMALNIAMLLTPDFVAHCEDGPLDADFTGSLGTLGSIPGFLAQLHQKVQLAAEVI
RAVQAEDEHERFLSPGTKPTLQAPVAPMHPRSSLRVGYPRLPDYEQEIRPLSPRLERLQDPANA
VVVVGYSELGPWGSARLRWEIESQGQWTSAGYVELAWLMNLIRHVNDESYVGWVDTQTGKPVRD
GEIQALYGDHIDNHTGIRPIQSTSYNPERMEVLQEVAVEEDLPEFEVSQLTADAMRLRHGANVS
IRPSGNPDACHVKLKRGAVILVPKTVPFVWGSCAGELPKGWTPAKYGIPENLIHQVDPVTLYTI
CCVAEAFYSAGITHPLEVFRHIHLSELGNFIGSSMGGPTKTRQLYRDVYFDHEIPSDVLQDTYL
NTPAAWVNMLLLGCTGPIKTPVGACATGVESIDSGYESIMAGKTKMCLVGGYDDLQEEASYGFA
QLKATVNVEEEIACGRQPSEMSRPMAESRAGFVEAHGCGVQLLCRGDIALQMGLPIYAVIASSA
MAADKIGSSVPAPGQGILSFSRERARSSMISVTSRPSSRSSTSSEVSDKSSLTSITSISNPAPR
AQRARSTTDMAPLRAALATWGLTIDDLDVASLHGTSTRGNDLNEPEVIETQMRHLGRTPGRPLW
AICQKSVTGHPKAPAAAWMLNGCLQVLDSGLVPGNRNLDTLDEALRSASHLCFPTRTVQLREVK
AFLLTSFGFGQKGGQVVGVAPKYFFATLPRPEVEGYYRKVRVRTEAGDRAYAAAVMSQAVVKIQ
TQNPYDEPDAPRIFLDPLARISQDPSTGQYRFRSDATPALDDDALPPPGEPTELVKGISSAWIE
EKVRPHMSPGGTVGVDLVPLASFDAYKNAIFVERNYTVRERDWAEKSADVRAAYASRWCAKEAV
FKCLQTHSQGAGAAMKEIEIEHGGNGAPKVKLRGAAQTAARQRGLEGVQLSISYGDDAVIAVAL
GLMSGAS
HexB
SEQ ID NO: 17
MGSVSREHESIPIQAAQRGAARICAAFGGQGSNNLDVLKGLLELYKRYGPDLDELLDVASNTLS
QLASSPAAIDVHEPWGFDLRQWLTTPEVAPSKEILALPPRSFPLNTLLSLALYCATCRELELDP
GQFRSLLHSSTGHSQGILAAVAITQAESWPTFYDACRTVLQISFWIGLEAYLFTPSSAASDAMI
QDCIEHGEGLLSSMLSVSGLSRSQVERVIEHVNKGLGECNRWVHLALVNSHEKFVLAGPPQSLW
AVCLHVRRIRADNDLDQSRILFRNRKPIVDILFLPISAPFHTPYLDGVQDRVIEALSSASLALH
SIKIPLYHTGTGSNLQELQPHQLIPTLIRAITVDQLDWPLVCRGLNATHVLDFGPGQTCSLIQE
LTQGTGVSVIQLTTQSGPKPVGGHLAAVNWEAEFGLRLHANVHGAAKLHNRMTTLLGKPPVMVA
GMTPTTVRWDFVAAVAQAGYHVELAGGGYHAERQFEAEIRRLATAIPADHGITCNLLYAKPTTF
SWQISVIKDLVRQGVPVEGITIGAGIPSPEVVQECVQSIGLKHISFKPGSFEAIHQVIQIARTH
PNFLIGLQWTAGRGGGHHSWEDFHGPILATYAQIRSCPNILLVVGSGFGGGPDTFPYLTGQWAQ
AFGYPCMPFDGVLLGSRMMVAREAHTSAQAKRLIIDAQGVGDADWHKSFDEPTGGVVTVNSEFG
QPIHVLATRGVMLWKELDNRVFSIKDTSKRLEYLRNHRQEIVSRLNADFARPWFAVDGHGQNVE
LEDMTYLEVLRRLCDLTYVSHQKRWVDPSYRILLLDFVHLLRERFQCAIDNPGEYPLDIIVRVE
ESLKDKAYRTLYPEDVSLLMHLFSRRDIKPVPFIPRLDERFETWFKKDSLWQSEDVEAVIGQDV
QRIFIIQGPMAVQYSISDDESVKDILHNICNHYVEALQADSRETSIGDVHSITQKPLSAFPGLK
VTTNRVQGLYKFEKVGAVPEMDVLFEHIVGLSKSWARTCLMSKSVFRDGSRLHNPIRAALQLQR
GDTIEVLLTADSEIRKIRLISPTGDGGSTSKVVLEIVSNDGQRVFATLAPNIPLSPEPSVVFCF
KVDQKPNEWTLEEDASGRAERIKALYMSLWNLGFPNKASVLGLNSQFTGEELMITTDKIRDFER
VLRQTSPLQLQSWNPQGCVPIDYCVVIAWSALTKPLMVSSLKCDLLDLLHSAISFHYAPSVKPL
RVGDIVKTSSRILAVSVRPRGTMLTVSADIQRQGQHVVTVKSDFFLGGPVLACETPFELTEEPE
MVVHVDSEVRRAILHSRKWLMREDRALDLLGRQLLFRLKSEKLFRPDGQLALLQVTGSVFSYSP
DGSTTAFGRVYFESESCTGNVVMDFLHRYGAPRAQLLELQHPGWTGTSTVAVRGPRRSQSYARV
SLDHNPIHVCPAFARYAGLSGPIVHGMETSAMMRRIAEWAIGDADRSRFRSWHITLQAPVHPND
PLRVELQHKAMEDGEMVLKVQAFNERTEERVAEADAHVEQETTAYVFCGQGSQRQGMGMDLYVN
CPEAKALWARADKHLWEKYGFSILHIVQNNPPALTVHFGSQRGRRIRANYLRMMGQPPIDGRHP
PILKGLTRNSTSYTFSYSQGLLMSTQFAQPALALMEMAQFEWLKAQGVVQKGARFAGHSLGEYA
ALGACASFLSFEDLISLIFYRGLKMQNALPRDANGHTDYGMLAADPSRIGKGFEEASLKCLVHI
IQQETGWFVEVVNYNINSQQYVCAGHFRALWMLGKICDDLSCHPQPETVEGQELRAMVWKHVPT
VEQVPREDRMERGRATIPLPGIDIPYHSTMLRGEIEPYREYLSERIKVGDVKPCELVGRWIPNV
VGQPFSVDKSYVQLVHGITGSPRLHSLLQQMA
StcJ
SEQ ID NO: 18
MTQKTIQQVPRQGLELLASTQDLAQLCYIYGEPAEGEDSTADESIINTPQCSTIPEVAVEPEVQ
PIPDTPLTAIFIIRALVARKLRRSETEIDPSRSIKELCGGKSTLQNELIGELGNEFQTSLPDRA
EDVSLADLDAALGEVSLGPTSVSLLQRVFTAKMPARMTVSNVRERLAEIWGLGFHRQTAVLVAA
LAAEPHSRLTSLEAAYQYWDGLNEAYGQSLGLFLRKAISQQAARSDDQGAQAIAPADSLGSKDL
ARKQYEALREYLGIRTPTTKQDGLDLADLQQKLDCWTAEFSDDFLSQISRRFDARKTRWYRDWW
NSARQELLTICQNSNVQWTDKMREHFVQRAEEGLVEIARAHSLAKPLVPDLIQAISLPPVVRLG
RLATMMPRTVVTLKGEIQCEEHEREPSCFVEFFSSWIQANNIRCTIQSNGEDLTSVFINSLVHA
SQQGVSFPNHTYLITGAGPGSIGQHIVRRLLTGGARVIVTTSREPLPAAAFFKELYSKCGNRGS
QLHLVPFNQASVVDCERLIGYIYDDLGLDLDAILPFAATSQVGAEIDGLDASNEAAFRLMLVNV
LRLVGFVVSQKRRRGISCRPTQVVLPLSPNHGILGGDGLYAESKRGLETLIQRFHSESWKEELS
ICGVSIGWTRSTGLMAANDLVAETAEKQGRVLTFSVDEMGDLISLLLTPQLATRCEDAPVMADF
SGNLSCWRDASAQLAAARASLRERADTARALAQEDEREYRCRRAGSTQEPVDQRVSLHLGFPSL
PEYDPLLHPDLVPADAVVVVGFAELGPWGSARIRWEMESRGCLSPAGYVETAWLMNLIRHVDNV
NYVGWVDGEDGKPVADADIPKRYGERILSNAGIRSLPSDNREVFQEIVLEQDLPSFETTRENAE
ALQQRHGDMVQVSTLKNGLCLVQLQHGATIRVPKSIMSPPGVAGQLPTGWSPERYGIPAEIVQQ
VDPVALVLLCCVAEAFYSAGISDPMEIFEHIHLSELGNFVGSSMGGVVNTRALYHDVCLDKDVQ
SDALQETYLNTAPAWVNMLYLGAAGPIKTPVGACATALESVDSAVESIKAGQTKICLVGGYDDL
QPEESAGFARMKATVSVRDEQARGREPGEMSRPTAASRSGFVESQGCGVQLLCRGDVALAMGLP
IYGIIAGTGMASDGIGRSVPAPGQGILTFAQEDAQNPAPSRTALARWGLGIDDITVASLHATST
PANDTNEPLVIQREMTHLGRTSGRPLWAICQKFVTGHPKAPAAAWMLNGCLQVLDTGLVPGNRN
ADDVDPALRSFSHLCFPIRSIQTDGIKAFLLNSCGFGQKEAQLVGVHPRYFLGLLSEPEFEEYR
TRRQLRIAGAERAYISAMMTNSIVCVQSHPPFGPAEMHSILLDPSARICLDSSTNSYRVTKAST
PVYTGFQRPHDKREDPRPSTIGVDTVTLSSFNAHENAIFLQRNYTERERQSLQLQSHRSFRSAV
ASGWCAKEAVFKCLQTVSKGAGAAMSEIEIVRVQGAPSVLHGDALAAAQKAGLDNIQLSLSYGD
DCVVAVALGVRKWCLWPLASIIR
StcK
SEQ ID NO: 19
MTPSPFLDAVDAGLSRLYACFGGQGPSNWAGLDELVHLSHAYADCAPIQDLLDSSARRLESQQR
SHTDRHFLLGAGSNYRPGSTTLLHPHHLPEDLALSPYSFPINTLLSLLHYAITAYSLQLDPGQL
RQKLQGAIGHSQGVFVAAAIAISHTDHGWPSFYRAADLALQLSFWVGLESHHASPRSILCANEV
IDCLENGEGAPSHLLSVTGLDINHLERLVRKLNDQGGDSLYISLINGHNKFVLAGAPHALRGVC
IALRSVKASPELDQSRVPFPLRRSVVDVQFLPVSAPYHSSLLSSVELRVTDAIGGLRLRGNDLA
IPVYCQANGSLRNLQDYGTHDILLTLIQSVTVERVNWPALCWAMNDATHVLSFGPGAVGSLVQD
VLEGTGMNVVNLSGQSMASNLSLLNLSAFALPLGKDWGRKYRPRLRKAAEGSAHASIETKMTRL
LGTPHVMVAGMTPTTCSPELVAAIIQADYHVEFACGGYYNRATLETALRQLSRSIPPHRSITCN
VIYASPKALSWQTQVLRRLIMEEGLPIDGITVGAGIPSPEVVKEWIDMLAISHIWFKPGSVDAI
DRVLTIARQYPTLPVGIQWTGGRAGGHHSCEDFHLPILDCYARIRNCENVILVAGSGFGGAEDT
WPYMNGSWSCKLGYAPMPFDGILLGSRMMVAREAKTSFAVKQLIVEAPGVKDDGNDNGAWAKCE
HDAVGGVISVTSEMGQPIHVLATRAMRLWKEFDDRFFSIRDPKRLKAALKQHRVEIINRLNNDF
ARPWFAQTDSSKPTEIEELSYRQVLRRLCQLTYVQHQARWIDSSYLSLVHDFLRLAQGRLGSGS
EAELRFLSCNTPIELEASFDAAYGVQGDQILYPEDVSLLINLFRRQGQKPVPFIPRLDADFQTW
FKKDSLWQSEDVDAVVDQDAQRVCIIQGPVAVRHSRVCDEPVKDILDGITEAHLKMMLKEAASD
NGYTWANQRDEKGNRLPGIETSQEGSLCRYYLVGPTLPSTEAIVEHLVGECAWGYAALSQKKVV
FGQNRAPNPIRDAFKPDIGDVIEAKYMDGCLREITLYHSLRRQGDPRAIRAALGLIHLDGNKVS
VTLLTRSKGKRPALEFKMELLGGTMGPLILKMHRTDYLDSVRRLYTDLWIGRDLPSPTSVGLNS
EFTGDRVTITAEDVNTFLAIVGQAGPARCRAWGTRGPVVPIDYAVVIAWTALTKPILLEALDAD
PLRLLHQSASTRFVPGIRPLHVGDTVTTSSRITERTITTIGQRVEISAELLREGKPVVRLQTTF
IIQRRPEESVSQQQFRCVEEPDMVIRVDSHTKLRVLMSRKWFLLDGPCSDLIGKILIFQLHSQT
VFDAAGAPASLQVSGSVSLAPSDTSVVCVSSVGTRIGRVYMEEEGFGANPVMDFLNRHGAPRVQ
RQPLPRAGWTGDDAASISFTAPAQSEGYAMVSGDTNPIHVCPLFSRFAGLGQPVVHGLHLSATV
RRILEWIIGDNERTRFCSWAPSFDGLVRANDRLRMEIQHFAMADGCMVVHVRVLKESTGEQVMH
AEAVLEQAQTTYVFTGQGTQERGMGMALYDTNAAARAVWDRAERHFRSQYGISLLHIVRENPTS
LTVNFGSRRGRQIRDIYLSMSDSDPSMLPGLTRDSRSYTFNYPSGLLMSTQFAQPALAVMEIAE
YAHLQAQGVVQTQAIFAGHSLGEYSSLGACTTIMPFESLLSLILYRGLKMQNTLPRNANGRTDY
GMVAADPSRIRSDFTEDRLIELVRLVSQATGVLLEVVNYNVHSRQYVCAGHVRSLWVLSHACDD
LSRSTSPNSPQTMSECIAHHIPSSCSVTNETELSRGRATIPLAGVDIPFHSQMLRGHIDGYRQY
LRHHLRVSDIKPEELVGRWIPNVTGKPFALDAPYIRLVQGVTQSRPLLELLRRVEENR
FAS alpha | FAS2
SEQ ID NO: 20
MRPEIEQELAHTLLVELLAYQFASPVRWIETQDVILAEKRTERIVEIGPADTLGGMARRTLASK
YEAYDAATSVQRQILCYNKDAKEIYYDVDPVEEETESAPEAAAAPPTSAAPAAAVVAAPAPAAS
APSAGPAAPVEDAPVTALDIVRTLVAQKLKKALSDVPLNKAIKDLVGGKSTLQNEILGDLGKEF
GSTPEKPEDTPLDELGASMQATFNGQLGKQSSSLIARLVSSKMPGGFNITAVRKYLETRWGLGP
GRQDGVLLLALTMEPASRIGSEPDAKVFLDDVANKYAANSGISLNVPTASGDGGASAGGMLMDP
AAIDALTKDQRALFKQQLEIIARYLKMDLRDGQKAFVASQETQKTLQAQLDLWQAEHGDFYASG
IEPSFDPLKARVYDSSWNWARQDALSMYYDIIFGRLKVVDREIVSQCIRIMNRSNPLLLEFMQY
HIDNCPTERGETYQLAKELGEQLIENCKEVLGVSPVYKDVAVPTGPQTTIDARGNIEYQEVPRA
SARKLEHYVKQMAEGGPISEYSNRAKVQNDLRSVYKLIRRQHRLSKSSQLQFNALYKDVVRALS
MNENQIMPQENGSTKKPGRNGSVRNGSPRAGKVETIPFLHLKKKNEHGWDYSKKLTGIYLDVLE
SAARSGLTFQGKNVLMTGAGAGSIGAEVLQGLISGGAKVIVTTSRYSREVTEYYQAMYARYGAR
GSQLVVVPFNQGSKQDVEALVDYIYDTKKGLGWDLDFIVPFAAIPENGREIDSIDSKSELAHRI
MLTNLLRLLGSVKAQKQANGFETRPAQVILPLSPNHGTFGNDGLYSESKLALETLFNRWYSENW
SNYLTICGAVIGWTRGTGLMSGNNMVAEGVEKLGVRTFSQQEMAFNLLGLMAPAIVNLCQLDPV
WADLNGGLQFIPDLKDLMTRLRTEIMETSDVRRAVIKETAIENKVVNGEDSEVLYKKVIAEPRA
NIKFQFPNLPTWDEDIKPLNENLKGMVNLDKVVVVTGFSEVGPWGNSRTRWEMEASGKFSLEGC
VEMAWIMGLIRHHNGPIKGKTYSGWVDSKTGEPVDDKDVKAKYEKYILEHSGIRLIEPELFKGY
DPKKKQLLQEIVIEEDLEPFEASKETAEEFKREHGEKVEIFEVLESGEYTVRLKKGATLLIPKA
LQFDRLVAGQVPTGWDARRYGIPEDIIEQVDPVTLFVLVCTAEAMLSAGVTDPYEFYKYVHLSE
VGNCIGSGIGGTHALRGMYKDRYLDKPLQKDILQESFINTMSAWVNMLLLSSTGPIKTPVGACA
TAVESVDIGYETIVEGKARVCFVGGFDDFQEEGSYEFANMKATSNAEDEFAHGRTPQEMSRPTT
TTRAGFMESQGCGMQLIMSAQLALDMGVPIYGIIALTTTATDKIGRSVPAPGQGVLTTARENPG
KFPSPLLDIKYRRRQLELRKRQIREWQESELLYLQEEAEAIKAQNPADFVVEEYLQERAQHINR
EAIRQEKDAQFSLGNNFWKQDSRIAPLRGALATWGLTVDEIGVASFHGTSTVANDKNESDVICQ
QMKHLGRKKGNALLGIFQKYLTGHPKGAAGAWMFNGCLQVLDSGLVPGNRNADNVDKVMEKFDY
IVYPSRSIQTDGIKAFSVTSFGFGQKGAQVIGIHPKYLYATLDRAQFEAYRAKVETRQKKAYRY
FHNGLVNNSIFVAKNKAPYEDELQSKVFLNPDYRVAADKKTSELKYPPKPPVATDAGSESTKAV
IESLAKAHATENSKIGVDVESIDSINISNETFIERILPASEQQYCQNAPSPQSSFAGRWSAKEA
VFKSLGVCSKGAGAPLKDIEIENDSNGAPTLHGVAAEAAKEAGVKHISVSISHSDMQAVAVAIS
QF
FAS beta | FAS1
SEQ ID NO: 21
MYGTSTGPQTGINTPRSSQSLRPLILSHGSLEFSFLVPTSLHFHASQLKDTFTASLPEPTDELA
QDDEPSSVAELVARYIGHVAHEVEEGEDDAHGTNQDVLKLTLNEFERAFMRGNDVHAVAATLPG
ITAKKVLVVEAYYAGRAAAGRPTKPYDSALFRAASDEKARIYSVLGGQGNIEEYFDELREVYNT
YTSFVDDLISSSAELLQSLSREPDANKLYPKGLNVMQWLREPDTQPDVDYLVSAPVSLPLIGLV
QLAHFAVTCRVLGKEPGEILERFSGTTGHSQGIVTAAAIATATTWESFHKAVANALTMLFWIGL
RSQQAYPRTSIAPSVLQDSIENGEGTPTPMLSIRDLPRTAVQEHIDMTNQHLPEDRHISISLVN
SARNFVVTGPPLSLYGLNLRLRKVKAPTGLDQNRVPFTQRKVRFVNRFLPITAPFHSQYLYSAF
DRIMEDLEDVEISPKSLTIPVYGTKTGDDLRAISDANVVPALVRMITHDPVNWEQTTAFPNATH
IVDFGPGGISGLGVLTNRNKDGTGVRVILAGSMDGTNAEVGYKPELFDRDEHSVKYAIDWVKEY
GPRLVKNATGQTFVDTKMSRLLGIPPIMVAGMTPTTVPWDFVAATMNAGYHIELAGGGYYNAKT
MTEAITKIEKAIPPGRGITVNLIYVNPRAMGWQIPLIGKLRADGVPIEGLTIGAGVPSIEVANE
YIETLGIKHIAFKPGSVDAIQQVINIAKANPKFPVILQWTGGRGGGHHSFEDFHQPILQMYSRI
RRHENIILVAGSGFGGAEDTYPYLSGNWSSRFGYPPMPFDGCLFGSRMMTAKEAHTSKNAKQAI
VDAPGLDDQDWEKTYKGAAGGVVTVLSEMGEPIHKLATRGVLFWHEMDQKIFKLDKAKRVPELK
KQRDYIIKKLNDDFQKVWFGRNSAGETVDLEDMTYAEVVHRMVDLMYVKHEGRWIDDSLKKLTG
DFIRRVEERFTTAEGQASLLQNYSELNVPYPAVDNILAAYPEAATQLINAQDVQHFLLLCQRRG
QKPVPFVPSLDENFEYWFKKDSLWQSEDLEAVVGQDVGRTCILQGPMAAKFSTVIDEPVGDILN
SIHQGHIKSLIKDMYNGDETTIPITEYFGGRLSEAQEDIEMDGLTISEDANKISYRLSSSAADL
PEVNRWCRLLAGRSYSWRHALFSADVFVQGHRFQTNPLKRVLAPSTGMYVEIANPEDAPKTVIS
VREPYQSGKLVKTVDIKLNEKGPIALTLYEGRTAENGVVPLTFLFTYHPDTGYAPIREVMDSRN
DRIKEFYYRIWFGNKDVPFYTPTTATFNGGRETITSQAVADFVHAVGNTGEAFVERPGKEVFAP
MDFAIVAGWKAITKPIFPRTIDGDLLKLVHLSNGFKMVPGAQPLKVGDVLDTTAQINSIINEES
GKIVEVCGTIRRDGKPIMHVTSQFLYRGAYTDFENTFQRKDEVPMQVHLASSRDVAILRSKEWF
RLDMDDVELLGQTLTFRLQSLIRFKNKNVFSQVQTMGQVLLELPTKEVIQVASVDYEAGTSHGN
PVIDYLQRNGTSIEQPVYFENPIPLSGKTPLVLRAPASNETYARVSGDYNPIHVSRVFSSYANL
PGTITHGMYTSAAVRSLVETWAAENNIGRVRGFHVSLVDMVLPNDLITVRLQHVGMIAGRKIIK
VEASNKETEDKVLLGEAEVEQPVTAYVFTGQGSQEQGMGMELYATSPVAKEVWDRPSFHWNYGL
SIIDIVKNNPKERTVHFGGPRGKAIRQNYMSMTFETVNADGTIKSEKIFKEIDETTTSYTYRSP
TGLLSATQFTQPALTLMEKASFEDMRSKGLVQRDSSFAGHSLGEYSALADLADVMLIESLVSVV
FYRGLTMQVAVERDEQGRSNYSMCAVNPSRISKTFNEQALQYVVGNISEQTGWLLEIVNYNVAN
MQYVAAGDLRALDCLTNLLNYLKAQNIDIPALMQSMSLEDVKAHLVNIIHECVKQTEAKPKPIN
LERGFATIPLKGIDVPFHSTFLRSGVKPFRSFLIKKINKTTIDPSKLVGKYIPNVTARPFEITK
EYFEDVYRLTNSPRIAHILANWEKYEEGTEGGSRHGGTTAASS

TABLE 4
HEXA HOMOLOGS

| Description | Ident | Accession |
|---|---|---|
| hypothetical protein [Aspergillus parasiticus SU-1] | 99% | KJK60794.1 |
| sterigmatocystin biosynthesis fatty acid synthase subunit alpha | | |
| [Aspergillus flavus AF70] | 98% | KOC17633.1 |
| fatty acid synthase alpha subunit [Aspergillus flavus NRRL3357] | 98% | XP_002379948.1 |
| HexA [Aspergillus flavus] | 98% | AAS90024.1 |
| unnamed protein product [Aspergillus oryzae RIB40] | 98% | XP_001821514.3 |
| sterigmatocystin biosynthesis fatty acid synthase subunit alpha | | |
| [Aspergillus arachidicola] | 97% | PIG79619.1 |
| sterigmatocystin biosynthesis fatty acid synthase subunit alpha | | |
| [Aspergillus bombycis] | 92% | XP_022391210.1 |
| sterigmatocystin biosynthesis fatty acid synthase subunit alpha | | |
| [Aspergillus nomius NRRL 13137] | 92% | XP_015404699.1 |

TABLE 5
HEXB HOMOLOGS

| Description | Ident | Accession |
|---|---|---|
| hypothetical protein [Aspergillus parasiticus SU-1] | 99% | KJK60796.1 |
| fatty acid synthase beta subunit [Aspergillus flavus NRRL3357] | 99% | XP_002379947.1 |
| HexB [Aspergillus flavus] | 99% | AAS90085.1 |
| unnamed protein product [Aspergillus oryzae RIB40] | 98% | XP_001821515.1 |
| fatty acid synthase beta subunit [Aspergillus flavus AF70] | 98% | KOC17632.1 |
| fatty acid synthase beta subunit [Aspergillus arachidicola] | 96% | PIG79622.1 |
| HexB [Aspergillus flavus] | 96% | AAS90002.1 |
| enoyl reductase domain of FAS1 [Aspergillus oryzae 3.042] | 98% | EIT81347.1 |
| fatty acid synthase beta subunit [Aspergillus bombycis] | 89% | XP_022391135.1 |
| HexB [Aspergillus nomius] | 90% | AAS90050.1 |
| fatty acid synthase beta subunit [Aspergillus nomius NRRL 13137] | 90% | XP_015404698.1 |

TABLE 6
FAS1 HOMOLOGS

| Description | Ident | Accession |
|---|---|---|
| fatty acid synthase, beta subunit [Aspergillus nidulans] | 100% | AAB41494.1 |
| hypothetical protein [Aspergillus nidulans FGSC A4] | 99% | XP_682677.1 |
| hypothetical protein [Aspergillus sydowii CBS 593.65] | 94% | OJJ52999.1 |
| Putative Fatty acid synthase beta subunit dehydratase [Aspergillus | | |
| calidoustus] | 94% | CEN62087.1 |
| hypothetical protein [Aspergillus versicolor CBS 583.65] | 93% | OJJ08968.1 |
| hypothetical protein [Aspergillus rambellii] | 91% | KKK18959.1 |
| hypothetical protein [Aspergillus ochraceoroseus] | 91% | KKK13726.1 |
| fatty acid synthase beta subunit dehydratase [Aspergillus terreus | | |
| NIH2624] | 91% | XP_001213436.1 |
| hypothetical protein [Aspergillus carbonarius ITEM 5010] | 89% | OOF94457.1 |
| hypothetical protein [Aspergillus turcosus] | 90% | OXN14637.1 |
| fatty acid synthase beta subunit [Aspergillus sclerotioniger CBS | | |
| 115572] | 89% | PWY96795.1 |
| fatty acid synthase beta subunit [Aspergillus heteromorphus CBS | | |
| 117.55] | 89% | XP_025394299.1 |
| fatty acid synthase beta subunit [Aspergillus sclerotiicarbonarius | | |
| CBS 121057] | 89% | PYI01270.1 |
| hypothetical protein [Aspergillus thermomutatus] | 90% | OXS11585.1 |

TABLE 7
FAS2 HOMOLOGS

| Description | Ident | Accession |
|---|---|---|
| RecName: Full = Fatty acid synthase subunit alpha; Includes: | 100% | P78615.1 |
| RecName: Full = Acyl carrier; Includes: RecName: Full = 3-oxoacyl- | | |
| [acyl-carrier-protein] reductase; AltName: Full = Beta-ketoacyl | | |
| reductase; Includes: RecName: Full = 3-oxoacyl-[acyl-carrier-protein] | | |
| synthase; AltName: Full = Beta-ketoacyl synthase | | |
| FAS2_PENPA Fatty acid synthase subunit alpha [Aspergillus | 99% | XP_682676.1 |
| nidulans FGSC A4] | | |
| TPA: Fatty acid synthase, alpha subunit | 99% | CBF87553.1 |
| [Source:UniProtKB/TrEMBL;Acc:P78615] [Aspergillus nidulans | | |
| FGSC A4] | | |
| hypothetical protein ASPVEDRAFT_144895 [Aspergillus versicolor | 93% | OJJ08967.1 |
| CBS 583.651 | | |
| Putative Fatty acid synthase subunit alpha reductase [Aspergillus | 93% | CEN62088.1 |
| calidoustus] | | |
| hypothetical protein ASPSYDRAFT_564317 [Aspergillus sydowii | 93% | OJJ52998.1 |
| CBS 593.65] | | |
| hypothetical protein BP01DRAFT_383520 [Aspergillus | 91% | XP_025430630.1 |
| saccharolyticus JOP 1030-1] | | |
| putative fatty acid synthase alpha subunit FasA [Aspergillus | 91% | PYI32058.1 |
| indologenus CBS 114.80] | | |
| hypothetical protein ASPCADRAFT_208136 [Aspergillus | 90% | OOF94458.1 |
| carbonarius ITEM 5010] | | |
| hypothetical protein ASPACDRAFT_79663 [Aspergillus aculeatus | 90% | XP_020055233.1 |
| ATCC 16872] | | |
| fatty acid synthase alpha subunit FasA [Aspergillus kawachii IFO | 91% | GAA92751.1 |
| 4308] | | |
| putative fatty acid synthase alpha subunit FasA [Aspergillus fijiensis | 90% | RAK72625.1 |
| CBS 313.89] | | |
| putative fatty acid synthase alpha subunit FasA [Aspergillus | 90% | XP_025498650.1 |
| aculeatinus CBS 121060] | | |
| putative fatty acid synthase alpha subunit FasA [Aspergillus | 90% | PYI15679.1 |
| violaceofuscus CBS 115571] | | |
| fatty acid synthase alpha subunit FasA [Aspergillus piperis CBS | 91% | XP_025520376.1 |
| 112811] | | |
| fatty acid synthase alpha subunit FasA [Aspergillus vadensis CBS | 91% | PYH66515.1 |
| 113365] | | |
| putative fatty acid synthase alpha subunit FasA [Aspergillus | 90% | XP_025442388.1 |
| brunneoviolaceus CBS 621.78] | | |
| fatty acid synthase alpha subunit FasA [Aspergillus neoniger CBS | 91% | XP_025476115.1 |
| 115656] | | |
| fatty acid synthase alpha subunit FasA [Aspergillus costaricaensis | 91% | RAK83984.1 |
| CBS 115574] | | |

### Fatty Acid and Fat Producing Genes

The production of fatty acids and fats in yeast may be increased by expressing rate limiting genes in the lipid biosynthesis pathway. Y. lipolytica naturally produces Acetyl-CoA. The overexpression of ACC1 increases the amount of Malonyl-CoA, which is the first step in fatty acid production. In certain embodiments, the one or more genetic modifications that result in increased production of fatty acids or fats comprise delta-9 stearoyl-CoA desaturase (SCD), Acetyl-CoA carboxylase (ACC1) and Diacylglyceride acyl-transferase (DGA1). The sequences for the native Y. lipolytica genes are shown herein, however the skilled person would understand that homologous genes may also be suitable. Examples of DGA1 homologs as shown in Table 8. In certain embodiments, SCD comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 22. In certain embodiments, SCD comprises a polypeptide that has at least 70%, 75%, 80%, 85%, 90%, 91%, 92%, 93%, 94%, 95%, 96%, 97%, 98%, 99% or 100% sequence identity to SEQ ID NO: 22. In certain embodiments, ACC1 comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 23. In certain embodiments, ACC1 comprises a polypeptide that has at least 70%, 75%, 80%, 85%, 90%, 91%, 92%, 93%, 94%, 95%, 96%, 97%, 98%, 99% or 100% sequence identity to SEQ ID NO: 23. In certain embodiments, DGA1 comprises a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 24. In certain embodiments, DGA1 comprises a polypeptide that has at least 70%, 75%, 80%, 85%, 90%, 91%, 92%, 93%, 94%, 95%, 96%, 97%, 98%, 99% or 100% sequence identity to SEQ ID NO: 24.

SCD, ACC1 and DGA1 may be overexpressed in yeast by adding extra copies of the genes driven by native or stronger promoters. Alternatively, native promoters may be substituted by stronger promoters such as TEFin, hp4d, hp8d and others, as would be appreciated by the person skilled in the art. The overexpression of SCD, ACC1 and DGA1 may be determined by quantitative PCR, Microarrays, or next generation sequencing technologies, such as RNA-seq. Alternatively, the product of increased enzyme levels will be increased production of fatty acids. Fatty acid production may be determined using chemical titration, thermometric titration, measurement of metal-fatty acid complexes using spectrophotometry, enzymatic methods or using a fatty acid binding protein.

Variants of the fatty acid and fat producing proteins, such as ACC1 retain the ability to produce malonyl-CoA from acetyl-CoA plus bicarbonate. For example, a variant of a fatty acid and fat producing protein, such as ACC1, must retain the ability to produce malonyl-CoA from acetyl-CoA plus bicarbonate with at least about 50%, at least about 60%, at least about 70%, at least about 80%, at least about 90%, or at least about 100% efficacy compared to the original sequence. In preferred embodiments, a variant of a fatty acid and fat producing protein, such as ACC1, has improved activity over the sequence from which it is derived in that the improved variant common cannabinoid protein has more than 110%, 120%, 130%, 140%, or and 150% improved activity in producing malonyl-CoA from acetyl-CoA plus bicarbonate, as compared to the sequence from which the improved variant is derived.

SCD
SEQ ID NO: 22
MVKNVDQVDLSQVDTIASGRDVNYKVKYTSGVKMSQGAYDDKGRHISEQPFTWANWHQHINWLN
FILVIALPLSSFAAAPFVSFNWKTAAFAVGYYMCTGLGITAGYHRMWAHRAYKAALPVRIILAL
FGGGAVEGSIRWWASSHRVHHRWTDSNKDPYDARKGFWFSHFGWMLLVPNPKNKGRTDISDLNN
DWVVRLQHKYYVYVLVFMAIVLPTLVCGFGWGDWKGGLVYAGIMRYTFVQQVTFCVNSLAHWIG
EQPFDDRRTPRDHALTALVTFGEGYHNFHHEFPSDYRNALIWYQYDPTKWLIWTLKQVGLAWDL
QTFSQNAIEQGLVQQRQKKLDKWRNNLNWGIPIEQLPVIEFEEFQEQAKTRDLVLISGIVHDVS
AFVEHHPGGKALIMSAVGKDGTAVFNGGVYRHSNAGHNLLATMRVSVIRGGMEVEVWKTAQNEK
KDQNIVSDESGNRIHRAGLQATRVENPGMSGMAA
ACC1
SEQ ID NO: 23
MRLQLRTLTRRFFSMASGSSTPDVAPLVDPNIHKGLASHFFGLNSVHTAKPSKVKEFVASHGGH
TVINKVLIANNGIAAVKEIRSVRKWAYETFGDERAISFTVMATPEDLAANADYIRMADQYVEVP
GGTNNNNYANVELIVDVAERFGVDAVWAGWGHASENPLLPESLAASPRKIVFIGPPGAAMRSLG
DKISSTIVAQHAKVPCIPWSGTGVDEVVVDKSTNLVSVSEEVYTKGCTTGPKQGLEKAKQIGFP
VMIKASEGGGGKGIRKVEREEDFEAAYHQVEGEIPGSPIFIMQLAGNARHLEVQLLADQYGNNI
SLFGRDCSVQRRHQKIIEEAPVTVAGQQTFTAMEKAAVRLGKLVGYVSAGTVEYLYSHEDDKFY
FLELNPRLQVEHPTTEMVTGVNLPAAQLQIAMGIPLDRIKDIRLFYGVNPHTTTPIDFDFSGED
ADKTQRRPVPRGHTTACRITSEDPGEGFKPSGGTMHELNFRSSSNVWGYFSVGNQGGIHSFSDS
QFGHIFAFGENRSASRKHMVVALKELSIRGDFRTTVEYLIKLLETPDFEDNTITTGWLDELISN
KLTAERPDSFLAVVCGAATKAHRASEDSIATYMASLEKGQVPARDILKTLFPVDFIYEGQRYKF
TATRSSEDSYTLFINGSRCDIGVRPLSDGGILCLVGGRSHNVYWKEEVGATRLSVDSKTCLLEV
ENDPTQLRSPSPGKLVKFLVENGDHVRANQPYAEIEVMKMYMTLTAQEDGIVQLMKQPGSTIEA
GDILGILALDDPSKVKHAKPFEGQLPELGPPTLSGNKPHQRYEHCQNVLHNILLGFDNQVVMKS
TLQEMVGLLRNPELPYLQWAHQVSSLHTRMSAKLDATLAGLIDKAKQRGGEFPAKQLLRALEKE
ASSGEVDALFQQTLAPLFDLAREYQDGLAIHELQVAAGLLQAYYDSEARFCGPNVRDEDVILKL
REENRDSLRKVVMAQLSHSRVGAKNNLVLALLDEYKVADQAGTDSPASNVHVAKYLRPVLRKIV
ELESRASAKVSLKAREILIQCALPSLKERTDQLEHILRSSVVESRYGEVGLEHRTPRADILKEV
VDSKYIVFDVLAQFFAHDDPWIVLAALELYIRRACKAYSILDINYHQDSDLPPVISWRFRLPTM
SSALYNSVVSSGSKTPTSPSVSRADSVSDFSYTVERDSAPARTGAIVAVPHLDDLEDALTRVLE
NLPKRGAGLAISVGASNKSAAASARDAAAAAASSVDTGLSNICNVMIGRVDESDDDDTLIARIS
QVIEDFKEDFEACSLRRITFSFGNSRGTYPKYFTFRGPAYEEDPTIRHIEPALAFQLELARLSN
FDIKPVHTDNRNIHVYEATGKNAASDKRFFTRGIVRPGRLRENIPTSEYLISEADRLMSDILDA
LEVIGTTNSDLNHIFINFSAVFALKPEEVEAAFGGFLERFGRRLWRLRVTGAEIRMMVSDPETG
SAFPLRAMINNVSGYVVQSELYAEAKNDKGQWIFKSLGKPGSMHMRSINTPYPTKEWLQPKRYK
AHLMGTTYCYDFPELFRQSIESDWKKYDGKAPDDLMTCNELILDEDSGELQEVNREPGANNVGM
VAWKFEAKTPEYPRGRSFIVVANDITFQIGSFGPAEDQFFFKVTELARKLGIPRIYLSANSGAR
IGIADELVGKYKVAWNDETDPSKGFKYLYFTPESLATLKPDTVVTTEIEEEGPNGVEKRHVIDY
IVGEKDGLGVECLRGSGLIAGATSRAYKDIFTLTLVTCRSVGIGAYLVRLGQRAIQIEGQPIIL
TGAPAINKLLGREVYSSNLQLGGTQIMYNNGVSHLTARDDLNGVHKIMQWLSYIPASRGLPVPV
LPHKTDVWDRDVTFQPVRGEQYDVRWLISGRTLEDGAFESGLFDKDSFQETLSGWAKGVVVGRA
RLGGIPFGVIGVETATVDNTTPADPANPDSIEMSTSEAGQVWYPNSAFKTSQAINDFNHGEALP
LMILANWRGFSGGQRDMYNEVLKYGSFIVDALVDYKQPIMVYIPPTGELRGGSWVVVDPTINSD
MMEMYADVESRGGVLEPEGMVGIKYRRDKLLDTMARLDPEYSSLKKQLEESPDSEELKVKLSVR
EKSLMPIYQQISVQFADLHDRAGRMEAKGVIREALVWKDARRFFFWRIRRRLVEEYLITKINSI
LPSCTRLECLARIKSWKPATLDQGSDRGVAEWFDENSDAVSARLSELKKDASAQSFASQLRKDR
QGTLQGMKQALASLSEAERAELLKGL
DGA1
SEQ ID NO: 24
MTIDSQYYKSRDKNDTAPKIAGIRYAPLSTPLLNRCETFSLVWHIFSIPTFLTIFMLCCAIPLL
WPFVIAYVVYAVKDDSPSNGGVVKRYSPISRNFFIWKLFGRYFPITLHKTVDLEPTHTYYPLDV
QEYHLIAERYWPQNKYLRAIISTIEYFLPAFMKRSLSINEQEQPAERDPLLSPVSPSSPGSQPD
KWINHDSRYSRGESSGSNGHASGSELNGNGNNGTTNRRPLSSASAGSTASDSTLLNGSLNSYAN
QIIGENDPQLSPTKLKPTGRKYIFGYHPHGIIGMGAFGGIATEGAGWSKLFPGIPVSLMTLTNN
FRVPLYREYLMSLGVASVSKKSCKALLKRNQSICIVVGGAQESLLARPGVMDLVLLKRKGFVRL
GMEVGNVALVPIMAFGENDLYDQVSNDKSSKLYRFQQFVKNFLGFTLPLMHARGVFNYDVGLVP
YRRPVNIVVGSPIDLPYLPHPTDEEVSEYHDRYIAELQRIYNEHKDEYFIDWTEEGKGAPEFRM
IE

TABLE 8
DGA1 HOMOLOGS

| Description | Ident | Accession |
|---|---|---|
| YALI0E32769p [Yarrowia lipolytica CLIB122] | 100% | XP_504700.1 |
| Diacylglycerol acyltransferase [Galactomyces candidus] | 44% | CDO57007.1 |
| hypothetical protein [Lipomyces starkeyi NRRL Y-11557] | 60% | ODQ70106.1 |
| DAGAT-domain-containing protein [Nadsonia fulvescens var. elongata | | |
| DSM 6958] | 60% | ODQ67305.1 |
| hypothetical protein [Tortispora caseinolytica NRRL Y-17796] | 65% | ODV90514.1 |
| diacylglycerol acyltransferase [Saitoella complicata NRRL Y-17804] | 60% | XP_019022950.1 |
| uncharacterized protein KUCA_T0000273600l [Kuraishia capsulata CBS | | |
| 1993] | 51% | XP_022458761.1 |
| diacylglycerol O-acyltransferas-like protein 2B [Meliniomyces bicolor E] | 55% | XP_024728739.1 |
| Diacylglycerol O-acyltransferase 1 [Hanseniaspora osmophila] | 57% | OEJ83128.1 |
| DAGAT-domain-containing protein [Ascoidea rubescens DSM 1968] | 49% | XP_020048004.1 |

In a second aspect of the present disclosure, there is provided method of producing at least one cannabinoid or cannabinoid precursor comprising contacting the yeast of the disclosure with a carbohydrate source under culture conditions and for a time sufficient to produce the at least one cannabinoid or cannabinoid precursor.

Specifically, examples of the culture conditions for producing at least one cannabinoid or cannabinoid precursor include a batch process and a fed batch or repeated fed batch process in a continuous manner, but are not limited thereto. Carbon sources that may be used for producing at least one cannabinoid or cannabinoid precursor may include sugars and carbohydrates such as glucose, sucrose, lactose, fructose, maltose, starch, xylose and cellulose; oils and fats such as soybean oil, sunflower oil, castor oil, coconut oil, chicken fat and beef tallow; fatty acids such as palmitic acid, stearic acid, oleic acid and linoleic acid; alcohols such as glycerol and ethanol; and organic acids such as gluconic acid, acetic acid, malic acid and pyruvic acid, but these are not limited thereto. These substances may be used alone or in a mixture. Nitrogen sources that may be used in the present disclosure may include peptone, yeast extract, meat extract, malt extract, corn steep liquor, defatted soybean cake, and urea or inorganic compounds, such as ammonium sulfate, ammonium chloride, ammonium phosphate, ammonium carbonate, and ammonium nitrate, but these are not limited thereto. These nitrogen sources may also be used alone or in a mixture. Phosphorus sources that may be used in the present disclosure may include potassium dihydrogen phosphate or dipotassium hydrogen phosphate, or corresponding sodium-containing salts, but these are not limited thereto. In addition, the culture medium may contain a metal salt such as magnesium sulfate or iron sulfate, which is may be required for the growth. Lastly, in addition to the above-described substances, essential growth factors such as amino acids and vitamins may be used. Such a variety of culture methods is disclosed, for example, in the literature (“Biochemical Engineering” by James M. Lee, Prentice-Hall International Editions, pp 138-176).

Basic compounds such as sodium hydroxide, potassium hydroxide, or ammonia, or acidic compounds such as phosphoric acid or sulfuric acid may be added to the culture medium in a suitable manner to adjust the pH of the culture medium. In addition, an anti-foaming agent such as fatty acid polyglycol ester may be used to suppress the formation of bubbles. In certain embodiments, the culture medium is maintained in an aerobic state, accordingly, oxygen or oxygen-containing gas (e.g., air) may be injected into the culture medium. The temperature of the culture medium may be usually 20° C. to 35° C., preferably 25° C. to 32° C., but may be changed depending on conditions. The culture may be continued until the maximum amount of a desired cannabinoid precursor or cannabinoid is produced, and it may generally be achieved within 5 hours to 160 hours. The cannabinoid precursor or cannabinoid may be released into the culture medium or contained in the yeast cells.

The method of the present disclosure for producing at least one cannabinoid or cannabinoid precursor may include a step of recovering the at least one cannabinoid or cannabinoid precursor from the microorganism or the medium. Methods known in the art, such as centrifugation, filtration, anion-exchange chromatography, crystallization, HPLC, etc., may be used for the method for recovering at least one cannabinoid or cannabinoid precursor from the microorganism or the culture, but the method is not limited thereto. The step of recovering may include a purification process. Specifically, following an overnight culture, 1 L cultures are pelleted by centrifugation, resuspended, washed in PBS and pelleted. The cells are lysed by either chemical or mechanical methods or a combination of methods. Mechanical methods can include a French Press or glass bead milling or other standard methods. Chemical methods can include enzymatic cell lysis, solvent cell lysis, or detergent based cell lysis. A liquid-liquid extraction of the cannabinoids is performed using the appropriate chemical solvent in which the cannabinoids are highly soluble and the solvent is not miscible in water. Examples include hexane, ethyl acetate, and cyclohexane, preferably solvents with straight or branched alkane chains (C5-C8) or mixtures thereof.

In certain embodiments, the at least one cannabinoid or cannabinoid precursor comprises CBGA, THCA, CBDA or CBCA. The production of one or more cannabinoid precursors or cannabinoids may be determined using a variety of methods as described herein. An example protocol for analysing CBDA is as follows:
1. Remove solvent from samples under vacuum.
2. Re-suspend dry samples in either 100 uL of dry hexane or dry ethyl acetate
3. Add 20 uL of N-Methyl-N-(trimethylsilyl)trifluoroacetamide (MSTFA)
4. Briefly mix
5. Heat solution to 60º C. for 10-15 minutes
6. GC-MS Method
   a. Instrument Agilent 6890-5975 GC-MS (Model Number: Agilent 19091S-433)
   b. Column HP-5MS 5% Phenyl Methyl Siloxane
   c. OVEN:
      i. Initial temp: 100° C. (On) Maximum temp: 300° C.
      ii. Initial time: 3.00 min Equilibration time: 0.50 min
      iii. Ramps:
         #Rate Final temp Final time
         1—30.00 280 1.00
         2—70.00 300 5.00
         3—0.0 (Off)
      iv. Post temp: 0° C.
      v. Post time: 0.00 min
      vi. Run time: 15.29 min

In a third aspect of the present disclosure, there is provided a cannabinoid precursor, cannabinoid or a combination thereof produced using the method of the second aspect. In certain embodiments, the at least one cannabinoid or cannabinoid precursor comprises CBGA, THCA, CBDA or CBCA.

### EXAMPLES

### Example 1: Vector Construction and Transformation

Y. lipolytica episomal plasmids comprise a centromere, origin and bacteria replicative backbone. Fragments for these regions were synthesized by Twist Bioscience and cloned to make an episomal parent vector pBM-pa. Plasmids were constructed by Gibson Assembly, Golden gate assembly, ligation or sequence-and ligation-independent cloning (SLIC). Genomic DNA isolation from bacteria (E. coli) and yeast (Yarrowia lipolytica) were performed using Wizard Genomic DNA purification kit according to manufacturer's protocol (Promega, USA). Synthetic genes were codon-optimized using GeneGenie or Genscript (USA) and assembled from gene fragments purchased from TwistBioscience. All the engineered Y. lipolytica strains were constructed by transforming the corresponding plasmids. All gene expression cassettes were constructed using a TEF intron promoter and synthesized short terminator. Up to six expression cassettes were cloned into episomal expression vectors through SLIC.

E. coli minipreps were performed using the Zyppy Plasmid Miniprep Kit (Zymo Research Corporation). Transformation of E. coli strains was performed using Mix & Go Competent Cells (Zymo research, USA). Transformation of Y. lipolytica with episomal expression plasmids was performed using the Zymogen Frozen EZ Yeast Transformation Kit II (Zymo Research Corporation), and spread on selective plates. Transformation of Y. lipolytica with linearized cassettes was performed using LiOAc method. Briefly, Y. lipolytica strains were inoculated from glycerol stocks directly into 10 ml YPD media, grown overnight and harvested at an OD600 between 9 and 15 by centrifugation at 1,000 g for 3 min. Cells were washed twice in sterile water. Cells were dispensed into separate microcentrifuge tubes for each transformation, spun down and resuspended in 1.0 ml 100 mM LiOAc. Cells were incubated with shaking at 30° C. for 60 min, spun down, resuspended in 90 ul 100 mM LiOAc and placed on ice. Linearized DNA (1-5 mg) was added to each transformation mixture in a total volume of 10 ul, followed by 25 ul of 50 mg/ml boiled salmon sperm DNA. Cells were incubated at 30° C. for 15 min with shaking, before adding 720 μl PEG buffer (50% PEG8000, 100 mM LiOAc, pH=6.0) and 45 μl 2 M Dithiothreitol. Cells were incubated at 30° C. with shaking for 60 min, heat-shocked for 10 min in a 39° C. water bath, spun down and resuspended in 1 ml sterile water. Cells (200 μl) were plated on appropriate selection plates.

### Example 2: Yeast Culture Conditions

E. coli strain DH10B was used for cloning and plasmid propagation. DH10B was grown at 37° C. with constant shaking in Luria-Bertani Broth supplemented with 100 mg/L of ampicillin for plasmid propagation. Y. lipolytica strains W29 was used as the base strain for all experiments. Y. lipolytica was cultivated at 30° C. with constant agitation. Cultures (2 ml) of Y. lipolytica used in large-scale screens were grown in a shaking incubator at speed 250 rpm for 1 to 3 days, and larger culture volumes were shaken in 50 ml flasks or fermented in a bioreactor.

For colony screening and cell propagation, Y. lipolytica grew on YPD liquid media contained 10 g/L yeast extract, 20 g/L peptone and 20 g/L glucose, or YPD agar plate with addition of 20 g/L of agar. Medium was often supplemented with 150 to 300 mg/L Hygromycin B or 250 to 500 mg/L nourseothricin for selection, as appropriate. For cannabinoid producing strains, modified YPD media with 0.1 to 1 g/L yeast extract was used for promoting lipid accumulation and often supplemented with 0.2 g/L and 5 g/L ammonium sulphate as alternative nitrogen source.

### Example 3: Cannabinoid Isolation

Y. lipolytica culture from the shaking flask experiment or bioreactor are pelleted and homogenized in acetonitrile followed by incubation on ice for 15 min. Supernatants are filtered (0.45 μm, Nylon) after centrifugation (13,100 g, 4° C., 20 min) and analyzed by HPLC-DAD. Quantification of products are based on integrated peak areas of the UV-chromatograms at 225 nm. Standard curves are generated for CBGA and THCA. The identity of all compounds can be confirmed by comparing mass and tandem mass spectra of each sample with coeluting standards analysed by Bruker Compact™ ESI-Q-TOF using positive ionization mode.

### Example 4: Gene Combinations

Embodiment 1: Y. lipolytica ERG20 comprising F88W and N119W substitutions; tHMGR; OLS: OAC; CBGAS; THCAS; HexA and HexB.

Embodiment 2: Y. lipolytica ERG20 comprising F88W and N119W substitutions; HMGR; OLS: OAC; NphB Q161A; THCAS; FAS1 I306A, M1251W and FAS2 G1250S.

Embodiment 3: S. cerevisiae ERG20 comprising a K197E substitution; OLS: OAC; NphB Q161A; CBDAS; StcJ and Stck.

Embodiment 4: Y. lipolytica ERG20 comprising a K189E substitution; HMGR; OLS: OAC; CBGAS; CBCAS; HexA and HexB.

Embodiment 5: Y. lipolytica ERG20 comprising a K189E substitution; tHMGR; OLS: OAC; CBGAS; CBDAS; StcJ and StcK.

The genetically modified yeast of the present disclosure enable the production of cannabinoid precursors and cannabinoids. The accumulation of fatty acids or fats in the yeast of at least 5% dry weight provides a storage location for the cannabinoid precursors and cannabinoids removed from the plasma membrane. This reduces the accumulation of cannabinoid precursors and cannabinoids in the plasma membrane, reducing membrane destabilisation and reducing the chances of cell death. Oily yeast such as Y. lipolytica can be engineered to have a fatty acid or fat (eg lipid) content above 80% dry weight, compared to 2-3% for yeast such as S. cerevisiae. Accordingly, cannabinoid precursor and cannabinoid production can be much higher in oily yeast, particularly oily yeast engineered to have a high fatty acid or fat (eg lipid) content.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope of the disclosure as set forth and defined by the following claims.

### REFERENCES

Ângela, C., Hansen, E. H., Kayser, O., Carlsen, S. and Stehle, F. 2017. Microorganism design for heterologous biosynthesis of cannabinoids. FEMS Yeast Research.
Bonitz, T., Alva, V., Saleh, O., Lupas, A. N. and Heide, L., 2011. Evolutionary relationships of microbial aromatic prenyltransferases. PloS one, 6(11), p. e27336.
Brown, D. W., Adams, T. H. and Keller, N. P., 1996. Aspergillus has distinct fatty acid synthases for primary and secondary metabolism. Proceedings of the National Academy of Sciences, 93(25), pp. 14873-14877.
Curran, K. A., Morse, N. J., Markham, K. A., Wagman, A. M., Gupta, A. and Alper, H. S., 2015. Short synthetic terminators for improved heterologous gene expression in yeast. ACS synthetic biology, 4(7), pp. 824-832.
Gao, S., Tong, Y., Zhu, L., Ge, M., Zhang, Y., Chen, D., Jiang, Y. and Yang, S., 2017. Iterative integration of multiple-copy pathway genes in Yarrowia lipolytica for heterologous β-carotene production. Metabolic engineering, 41, pp. 192-201.
Gajewski, J., Pavlovic, R., Fischer, M., Boles, E. and Grininger, M., 2017. Engineering fungal de novo fatty acid synthesis for short chain fatty acid production. Nature Communications, 8, p. 14650.
Ghorai, N., Chakraborty, S., Gucchait, S., Saha, S. K. and Biswas, S., 2012. Estimation of total Terpenoids concentration in plant tissues using a monoterpene, Linalool as standard reagent. Protocol Exchange, 5.
Hitchman, T. S., Schmidt, E. W., Trail, F., Rarick, M. D., Linz, J. E. and Townsend, C. A., 2001. Hexanoate synthase, a specialized type I fatty acid synthase in aflatoxin B1 biosynthesis. Bioorganic chemistry, 29(5), pp. 293-307.
Kampranis, S. C. and Makris, A. M. 2012. Developing a yeast cell factory for the production of terpenoids. Computational and structural biotechnology journal 3, p. e201210006.
Kuzuyama, T., Noel, J. P. and Richard, S. B., 2005. Structural basis for the promiscuous biosynthetic prenylation of aromatic natural products. Nature, 435(7044), p. 983.
Lange, K., Schmid, A. and Julsing, M. K. 2016. Δ9-Tetrahydrocannabinolic acid synthase: The application of a plant secondary metabolite enzyme in biocatalytic chemical synthesis. Journal of Biotechnology 233, pp. 42-48.
MacPherson, M. and Saka, Y., 2016. Short synthetic terminators for assembly of transcription units in vitro and stable chromosomal integration in yeast S. cerevisiae. ACS synthetic biology, 6(1), pp. 130-138.
Muntendam, R. (2015). Metabolomics and bioanalysis of terpenoid derived secondary metabolites: Analysis of Cannabis sativa L. metabolite production and prenylases for cannabinoid production [Groningen].
Poulos, J. L. and Farnia, A. 2016. Patent US20160010126—Production of cannabinoids in yeast—Google Patents. Available at: https://www.google.com/patents/US20160010126 [Accessed: 5 May 2017].
Qiao, K., Imam Abidi, S. H., Liu, H., Zhang, H., Chakraborty, S., Watson, N., Kumaran Ajikumar, P. and Stephanopoulos, G. 2015. Engineering lipid overproduction in the oleaginous yeast Yarrowia lipolytica. Metabolic Engineering 29, pp. 56-65.
Zhao, J., Bao, X., Li, C., Shen, Y. and Hou, J. 2016. Improving monoterpene geraniol production through geranyl diphosphate synthesis regulation in Saccharomyces cerevisiae. Applied Microbiology and Biotechnology 100(10), pp. 4561-4571.
Zhuang, X. U. N. Engineering Novel Terpene Production Platforms In The Yeast Saccharomyces Cerevisiae.
Zirpel, B., Degenhardt, F., Martin, C., Kayser, O. and Stehle, F. 2017. Engineering yeasts as platform organisms for cannabinoid biosynthesis. Journal of Biotechnology.
Zirpel, B., Stehle, F. and Kayser, O. 2015. Production of Δ9-tetrahydrocannabinolic acid from cannabigerolic acid by whole cells of Pichia (Komagataella) pastoris expressing Δ9-tetrahydrocannabinolic acid synthase from Cannabis sativa L. Biotechnology Letters 37(9), pp. 1869-1875.

1. A genetically modified yeast comprising:
(a) one or more GPP producing genes and optionally, one or more GPP pathway genes;
(b) two or more olivetolic acid producing genes;
(c) one or more cannabinoid precursor or cannabinoid producing genes;
(d) one or more Hexanoyl-CoA producing genes, and
(e) at least 5% dry weight of fatty acids or fats.

2. The yeast of claim 1, wherein the one or more GPP producing gene comprises at least one of:
a) a mutated farnesyl diphosphate synthase;
b) a mutated S. cerevisiae ERG₂₀ comprising a K197E substitution;
c) a double mutated S. cerevisiae ERG₂₀ comprising F96W and N127W substitutions;
d) a mutated Y. lipolytica ERG₂₀ comprising a K189E substitution;
e) a double mutated Y. lipolytica ERG₂₀ comprising F88W and N119W substitutions;
f) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 1-4;
g) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 1-4; or
h) any combination of (a)-(g).

3. The yeast of claim 1, wherein the one or more GPP producing genes is selected from a GPP pathway gene.

4. The yeast of claim 3, wherein the GPP pathway gene is selected from:
a) a hydroxymethylglutaryl-CoA reductase (HMGR);
b) a truncated Hydroxymethylglutaryl-CoA reductase (tHMGR);
c) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 5-6;
d) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 5-6; or
e) any combination of (a)-(d);

5. The yeast of claim 1, wherein the two or more olivetolic acid producing genes comprise: at least one of (a)-(d) and at least one of (e)-(h);
wherein (a)-(d) comprise:
a) an olivetol synthase (OLS);
b) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 7;
c) a polypeptide that has at least 70%, 75%, 80%, 85%, 90%, 91%, 92%, 93%, 94%, 95%, 96%, 97%, 98%, 99% or 100% sequence identity to SEQ ID NO: 7; or
d) any combination of (a)-(c);
and wherein (e)-(h) comprise:
e) an olivetolic acid cyclase (OAC);
f) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 8;
g) a polypeptide that has at least 70%, 75%, 80%, 85%, 90%, 91%, 92%, 93%, 94%, 95%, 96%, 97%, 98%, 99% or 100% sequence identity to SEQ ID NO: 8; or
h) any combination of (e)-(g).

6. The yeast of claim 1, wherein the one or more cannabinoid precursor or cannabinoid producing genes comprise at least one of:
a) a soluble aromatic prenyltransferase;
b) a cannabigerolic acid synthase (CBGAS);
c) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 9-12;
d) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 9-12; or
e) any combination of (a)-(d);
alone or in combination with at least one of:
f) a tetrahydrocannabinolic acid synthase (THCAS);
g) a cannabidiolic acid synthase (CBDAS);
h) a cannabichromenic acid synthase (CBCAS);
i) a polynucleotide encoding a polypeptide that has at least 70%, 75%, 80%, 85%, 90%, 91%, 92%, 93%, 94%, 95%, 96%, 97%, 98%, 99% or 100% sequence identity to any one of SEQ ID NOS: 13-15;
j) a polypeptide that has at least 70%, 75%, 80%, 85%, 90%, 91%, 92%, 93%, 94%, 95%, 96%, 97%, 98%, 99% or 100% sequence identity to any one of SEQ ID NOS: 13-15; or
k) any combination of (f)-(j).

7. The yeast of claim 6, wherein the soluble aromatic prenyltransferase is NphB from Streptomyces sp. strain CL₁₉₀.

8. The yeast of claim 1, wherein the one or more Hexanoyl-CoA producing genes comprise at least one of:
a) a hexanoyl-CoA synthase;
b) HexA and HexB;
c) StcJ and Stck;
d) a mutated FAS1 and a mutated FAS2;
e) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 16 and a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 17;
f) a polypeptide that has at least 70%, 75%, 80%, 85%, 90%, 91%, 92%, 93%, 94%, 95%, 96%, 97%, 98%, 99% or 100% sequence identity to SEQ ID NO: 16 and a polypeptide that has at least 70%, 75%, 80%, 85%, 90%, 91%, 92%, 93%, 94%, 95%, 96%, 97%, 98%, 99% or 100% sequence identity to SEQ ID NO: 17;
g) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 18 and a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 19;
h) a polypeptide that has at least 70%, 75%, 80%, 85%, 90%, 91%, 92%, 93%, 94%, 95%, 96%, 97%, 98%, 99% or 100% sequence identity to SEQ ID NO: 18 and a polypeptide that has at least 70%, 75%, 80%, 85%, 90%, 91%, 92%, 93%, 94%, 95%, 96%, 97%, 98%, 99% or 100% sequence identity to SEQ ID NO: 19;
i) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 20 and a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 21;
j) a polypeptide that has at least 70%, 75%, 80%, 85%, 90%, 91%, 92%, 93%, 94%, 95%, 96%, 97%, 98%, 99% or 100% sequence identity to SEQ ID NO: 20 and a polypeptide that has at least 70%, 75%, 80%, 85%, 90%, 91%, 92%, 93%, 94%, 95%, 96%, 97%, 98%, 99% or 100% sequence identity to SEQ ID NO: 21; or
k) any combination of (a)-(j).

9. The yeast of claim 8, wherein the mutated FAS1 and FAS2 genes comprise the genetic modifications selected from:
a) FAS1 I306A and FAS2 G1250S;
b) FAS1 I306A, M1251W and FAS2 G1250S; or
c) FAS1 I306A, R1834K and FAS2 G1250S.

10. The yeast of claim 1, wherein the yeast comprises at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% dry weight of fatty acids or fats.

11. The yeast of claim 1, wherein the yeast is genetically modified to produce at least 5%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, or at least 25% dry weight of fatty acids or fats.

12. The yeast of claim 11, wherein the yeast further comprises genetic modifications that increase the production of fatty acids or fats.

13. The yeast of claim 1, wherein the yeast is oleaginous.

14. The yeast of claim 12, wherein the yeast is selected from the genera Rhodosporidium, Rhodotorula, Yarrowia, Cryptococcus, Candida, Lipomyces and Trichosporon.

15. The yeast of claim 1, wherein the yeast is a Yarrowia lipolytica, a Lipomyces starkey, a Rhodosporidium toruloides, a Rhodotorula glutinis, a Trichosporon fermentans or a Cryptococcus curvatus.

16. A method of producing at least one cannabinoid or cannabinoid precursor comprising contacting the yeast of claim 1 with a carbohydrate source under culture conditions and for a time sufficient to produce the at least one cannabinoid or cannabinoid precursor.

17. The method of claim 17, wherein the at least one cannabinoid or cannabinoid precursor comprises CBGA, THCA, CBDA or CBCA.

18. A cannabinoid precursor, cannabinoid or a combination thereof produced using the method of claim 17.

19. The yeast of claim 12, wherein the genetic modifications that increase the production of fatty acids or fats comprise at least one of:
a) a delta-9 stearoyl-CoA desaturase (SCD);
b) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 22;
c) a polypeptide that has at least 70%, 75%, 80%, 85%, 90%, 91%, 92%, 93%, 94%, 95%, 96%, 97%, 98%, 99% or 100% sequence identity to SEQ ID NO: 22
d) Acetyl-CoA carboxylase (ACC1);
e) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 23;
f) a polypeptide that has at least 70%, 75%, 80%, 85%, 90%, 91%, 92%, 93%, 94%, 95%, 96%, 97%, 98%, 99% or 100% sequence identity to SEQ ID NO: 23;
g) Diacylglyceride acyl-transferase (DGA1);
h) a polynucleotide encoding a polypeptide that has at least 70%, 75%, 80%, 85%, 90%, 91%, 92%, 93%, 94%, 95%, 96%, 97%, 98%, 99% or 100% sequence identity to SEQ ID NO: 24;
i) a polypeptide that has at least 70%, 75%, 80%, 85%, 90%, 91%, 92%, 93%, 94%, 95%, 96%, 97%, 98%, 99% or 100% sequence identity to SEQ ID NO:24; or
j) any combination of (a)-(i).
